(19) United States
(12) Patent Application Publication
(10) Pub. No.: US 2024/0260874 A1
(43) Pub. Date: Aug. 8, 2024

(54) OBTAINING URINE CHARACTERISTICS TO PROVIDE FLUID THERAPY, AND ASSOCIATED SYSTEMS, DEVICES, AND METHODS

(71) Applicant: REPRIEVE CARDIOVASCULAR, INC., Milford, MA (US)

(72) Inventors: Andrew Halpert, Brookline, MA (US); Eric Conley, York, ME (US); Jeffrey Testani, New Haven, CT (US)

(21) Appl. No.: 18/434,540

(22) Filed: Feb. 6, 2024

Related U.S. Application Data
(60) Provisional application No. 63/483,494, filed on Feb. 6, 2023.

(51) Int. Cl. A61B 5/20; A61M 5/142; G01N 33/493

(52) U.S. Cl. CPC A61B 5/207; A61M 5/142; G01N 33/493

(57) ABSTRACT

Embodiments of the present technology can manage a patient's fluid removal based on one or more of the patient's urine output, urine conductivity, urine sodium concentration, and/or urine oxygen content. For example, in some embodiments the patient's urine sodium concentration (and/or an indication thereof including, e.g., the patient's urine conductivity) can be compared to one or more urine sodium concentration thresholds (e.g., a high urine sodium concentration threshold and/or a low urine sodium concentration threshold) to determine one or more adjustments to the patient's therapy such as, e.g., a diuretic dosage rate, a hydration fluid infusion rate, a hydration fluid matching percentage, etc. Additionally or alternatively, the patient's urine oxygen content can be compared to one or more urine oxygen content thresholds (e.g., a high urine oxygen content threshold and/or a low urine oxygen content threshold) to determine one or more adjustments to the patient's therapy.

### CROSS-REFERENCE TO RELATED APPLICATION(S)

The present application claims priority to U.S. Provisional App. No. 63/483,494, filed Feb. 6, 2023, the disclosure of which is incorporated herein by reference in its entirety.

### TECHNICAL FIELD

The present disclosure generally relates to medical devices and, in particular, to obtaining urine characteristics to provide fluid therapy, and associated systems, devices, and methods.

### BACKGROUND

Human physiological systems seek to naturally maintain a balance between fluid intake and fluid excretion. An imbalance in fluid intake and excretion rates may cause the body to retain excess amounts of fluid, also known as fluid overload. Fluid overload can be caused by acute decompensated heart failure (ADHF), chronic heart failure (CHF), or other conditions in which insufficient fluid is excreted. Patients exhibiting fluid overload may suffer from shortness of breath (dyspnea), edema, hypertension, and other undesirable medical conditions.

To treat fluid overload, patients are typically administered a diuretic drug which induces and/or increases urine production, thus reducing the amount of fluid and sodium in the body. The rate of urine output may be carefully monitored and/or controlled for safety reasons, e.g., to avoid placing undue stress on the patient's kidneys. Different patients may respond differently to treatment, such that the same diuretic type and/or dosage may produce drastically different urine output rates. However, conventional systems and methods for treating fluid overload may not be capable of accurately monitoring a patient's urine output and/or urine characteristics to respond to changes in urine output.

### BRIEF DESCRIPTION OF THE DRAWINGS

Features, aspects, and advantages of the presently disclosed technology may be better understood with regard to the following drawings.

FIG. 1 is a partially schematic view of a fluid management system, configured in accordance with embodiments of the present technology.

FIG. 2 is a flow diagram of a method for treating a patient, configured in accordance with embodiments of the present technology.

FIG. 3 is a partially schematic illustration of a urine flow cartridge, configured in accordance with embodiments of the present technology.

FIG. 4 is a partially schematic illustration of a urine flow cartridge, configured in accordance with embodiments of the present technology.

FIGS. 5A-5I illustrate a representative example of a urine collection system, in accordance with embodiments of the present technology.

FIGS. 6-9 are block diagrams illustrating methods of providing outputs associated with a patient's fluid therapy, configured in accordance with embodiments of the present technology.

A person skilled in the relevant art will understand that the features shown in the drawings are for purposes of illustrations, and variations, including different and/or additional features and arrangements thereof, are possible.

### DETAILED DESCRIPTION

### I. OVERVIEW

The present technology is directed to systems for managing (e.g., increasing or decreasing) a patient's urine output based at least in part on characteristics of the patient's urine. Such characteristics can include urine output (e.g., a volume and/or a rate of urine output), urine conductivity, urine sodium concentration (e.g., based on or at least in part on a conductivity of the patient's urine), urine temperature, and/or urine oxygen content (e.g., a partial pressure of oxygen in the patient's urine). Embodiments of the present technology relate to infusing diuretic and/or hydration fluid to increase or optimize urine output from the patient. While a standard treatment protocol can be effective for most patients, some patients can have unique conditions and/or have abnormal responses to the standard treatment protocols that prevent or inhibit optimal therapy. As an example, certain patients may not react to some diuretics and/or may have underlying conditions (e.g., low or high blood pressure) which limit their urine output rates, or make treatment to achieve maximum urine output rates more difficult. For such patients, additional steps or protocols may be necessary to increase urine output and relieve fluid overload conditions. These additional steps or protocols can be based on data associated with a patient receiving therapy, such as the patient's response to the received therapy (e.g., the administered diuretic and/or hydration fluid), and/or on historical treatment data including the treatment responses of one or more other patients. Accordingly, embodiments of the present technology are expected to optimize and/or customize all or a subset of a diuretic therapy to an individual patient's physiology, for example, to maximize decongestion and/or minimize clinical sequelae.

As described elsewhere herein, embodiments of the present technology can manage a patient's fluid removal based on one or more of the patient's urine output, urine conductivity, urine sodium concentration, and/or urine oxygen content. For example, in some embodiments the patient's urine sodium concentration (and/or an indication thereof including, e.g., the patient's urine conductivity) can be compared to one or more urine sodium concentration thresholds (e.g., a high urine sodium concentration threshold and/or a low urine sodium concentration threshold) to determine one or more adjustments to the patient's therapy such as, e.g., a diuretic dosage rate, a hydration fluid infusion rate, a hydration fluid matching percentage, etc. Additionally or alternatively, the patient's urine oxygen content can be compared to one or more urine oxygen content thresholds (e.g., a high urine oxygen content threshold and/or a low urine oxygen content threshold) to determine one or more adjustments to the patient's therapy. In some embodiments, the one or more adjustments to the patient's therapy can be based on a combination of two or more of the characteristics described herein including urine output rate, urine conductivity, urine sodium concentration, urine temperature, and urine oxygen content. Such combinations can include the urine output and the urine conductivity, the urine output and the urine oxygen content, the urine conductivity and the urine oxygen content, and/or the urine output, urine conductivity, and the urine oxygen content.

These and other embodiments of the present technology can manage a patient's fluid removal based on the measured urine output and the physician's estimated excess fluid volume. For example, in some embodiments if a patient's urine output drops below a pre-defined rate and the patient has lost 80% or more of the estimated excess fluid volume or less than 1 L of estimated excess fluid remains to be removed from the patient, then the system may determine that therapy should be stopped (e.g., automatically stopped) immediately or after a period of time (e.g., one hour). Alternatively, if a patient's urine output drops below a pre-defined rate and less than 80% of the estimated excess fluid volume has been removed and/or more than 1 L of estimated excess fluid remains to be removed from the patient, then the system may determine that it is necessary to take steps to increase urine production. In such embodiments, the system may recommend (e.g., via software, labeling, etc.) infusing a second diuretic in addition to a first diuretic already being infused, and/or adjusting a rate of hydration fluid infusion. In doing so, embodiments of the present technology can advantageously manage a patient's urine output by ceasing one or more aspects of fluid therapy (e.g., diuretic infusion) for instances of sufficient fluid loss and improving fluid therapy by increasing urine output for instances of insufficient fluid loss.

The headings provided herein are for convenience only and are not intended to limit or interpret the scope or meaning of the technology.

### II. FLUID MANAGEMENT SYSTEMS AND METHODS

The present technology is generally directed to systems, devices, and associated methods for fluid therapy based on patient data, including managing fluid levels of the patient based at least partially in response to data received from the patient before and/or during the fluid therapy. In some embodiments, the systems, devices, and methods described herein are used to treat a patient for fluid overload. To treat fluid overload, patients can be administered a diuretic drug which induces and/or increases urine production. For example, loop diuretics are diuretics that act at the ascending limb of the loop of Henle in the kidney, and include bumetanide (Bumex®), ethacrynic acid (Edecrin®), furosemide (Lasix®), torsemide (Demadex®), thiazide and thiazide-like diuretics (e.g., chlorothiazide, metolazone), potassium-sparing diuretics (e.g., amiloride, spironolactone), carbonic anhydrase inhibitors (e.g., acetazolamide), Vaptans (e.g., Conivaptan), SGLT2 inhibitors, and osmotic diuretics (e.g., mannitol). Diuretics can be given orally as a pill or as an intravenous (IV) injection. IV diuretics can be used when oral diuretics are no longer effective and/or able to be absorbed.

The short-term effects of diuretics on a patient's urine production may be difficult to predict, particularly at early stages of treatment. For example, one patient may produce much less urine than expected for a given dose of diuretic, while another patient administered the same dose may produce very large amounts of urine. Low urine production can prolong treatment time and/or reduce treatment efficacy, while high urine production can raise concerns of hypotension, hypovolemia, electrolyte imbalance (e.g., hypokalemia), and/or vital organ damage. High doses of a diuretic, regardless of the urine response, can also raise concerns about ototoxicity. Due to these uncertainties, physicians typically initially prescribe a conservative (e.g., low) diuretic dosage and wait a few hours before considering whether to increase the dosage. If the physician determines that a higher diuretic dosage is needed, the physician may slowly and incrementally increase the dosage until the patient's urine output reaches the desired level and/or rate. However, this approach can prolong the time the patient remains in the fluid overloaded condition, which can exacerbate the patient's underlying clinical state. For example, conservative treatment procedures can require hours or even days before the patient's urine output is sufficiently high to cause significant fluid loss and relieve the fluid overload condition. The patient may be hospitalized for several days (e.g., 4-5 days), which can be expensive and burdensome. Additionally, the long-term treatment efficacy may be limited, such that approximately 25% of patients are readmitted for fluid overload within 30 days.

To overcome these and other challenges, the present technology provides systems, and associated devices and methods, for managing a patient's fluid levels. In some embodiments, the present technology can (i) improve efficacy, safety, and quality of fluid management treatment, (ii) improve resource management in hospitals and other clinical settings, (iii) quickly assess if a patient is diuretic resistant, and/or (iv) increase diuretic efficiency (the amount of urine and/or excreted electrolytes (e.g., sodium) obtained over a given time per mg of diuretic infused intravenously). The embodiments described herein can increase net removal of fluid and/or electrolytes (e.g., sodium and/or chloride), and can also treat fluid overload conditions in a more efficient manner (e.g., shorter timeframe and/or higher net fluid loss).

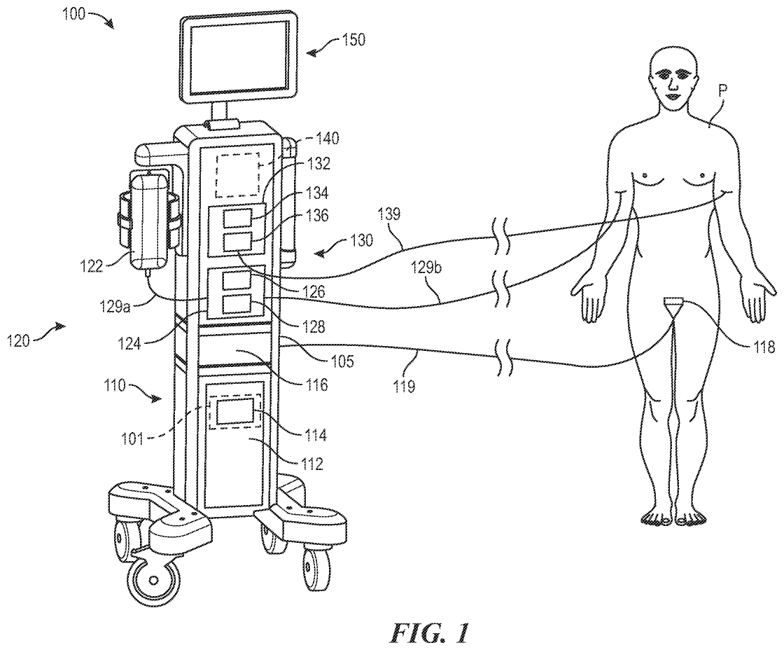

FIG. 1 is a partially schematic illustration of a fluid management system 100 (“system 100”) for monitoring urine output and/or control fluid infusion into a patient P, in accordance with embodiments of the present technology. The system 100 includes a urine collection and monitoring system 110 (“urine system 110”), an automated hydration fluid infusion system 120 (“hydration system 120”), an automated diuretic infusion system 130 (“diuretic system 130”), a controller or control system 140 (“controller 140”), and a display or input/output unit 150 (“display 150”). The controller 140 can be operably coupled to each of the urine system 110, hydration system 120, diuretic system 130, and/or display 150. The system 100 can further include a console or structure 105 (“console 105”) that incorporates, houses, and/or otherwise supports all or portions of the urine system 110, hydration system 120, diuretic system 130, the controller 140, and/or the display 150.

The urine system 110 is configured to collect urine from the patient P and/or monitor the patient's urine output (e.g., urine output amount and/or rates). The urine system 110 can include one or more collection containers 112 (“container 112”) configured to hold urine, such as a disposable bag or other collection device. The container 112 can be fluidly coupled to the patient P via a fluid line 119 (e.g., a tubing line). The fluid line 119 can include a single continuous fluid line or multiple fluid line portions (e.g., first, second, third, etc. fluid line portions; proximal and distal fluid line portions, etc.) in fluid communication with one another. The fluid line 119 can be connectable to a disposable catheter 118 (e.g., a Foley catheter, Texas Condom catheter, PureWick catheter, etc.) placed in or otherwise connected to the bladder of the patient P.

In some embodiments, urine flow through the fluid line 119 is driven by the patient's urine production, gravity (e.g., the bladder of the patient P is positioned higher than the container 112), and/or a siphon effect between the patient's bladder and the container 112. In other embodiments, the urine system 110 can also include a pump (not shown) operably coupled to the fluid line 119 for actuating urine flow through the fluid line 119 and into the container 112. The pump can be or include any device suitable for pumping fluid, such as a peristaltic pump. The pump can be used to initiate urine flow from the patient's body at the start of the procedure. The pump can also be used to clear air locks and/or other obstructions from the fluid line 119.

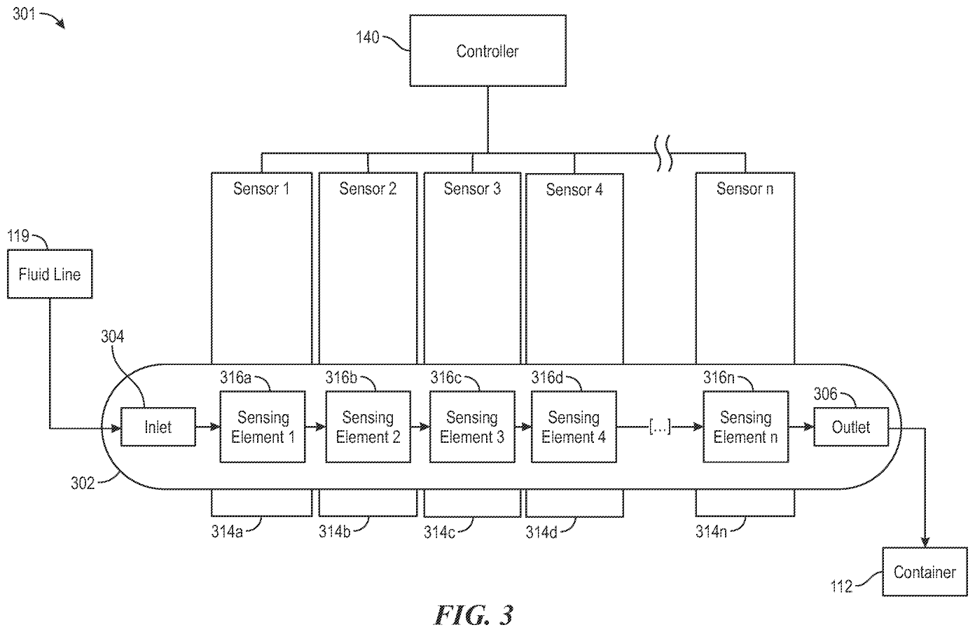

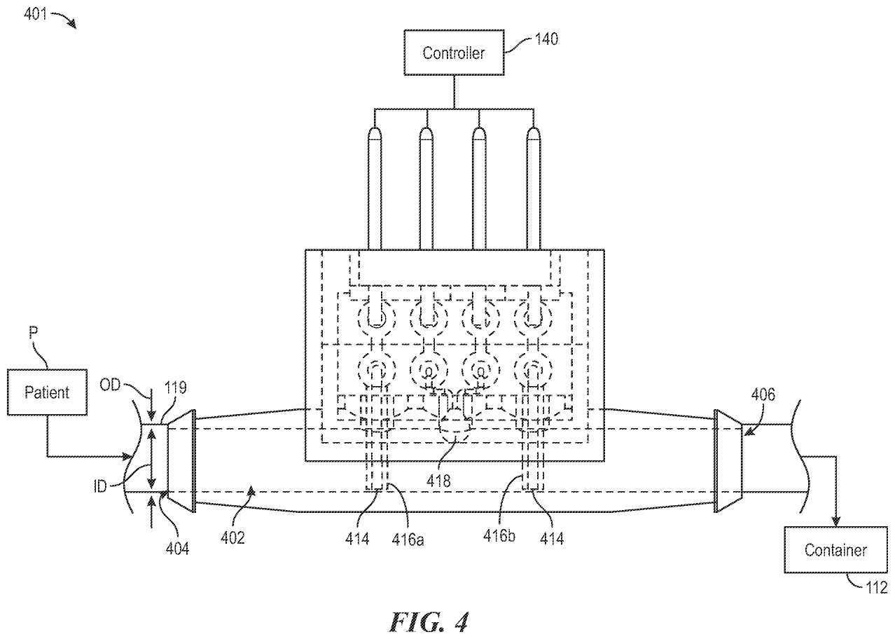

The urine system 110 can include one or more sensors 114 (“sensor(s) 114”) configured to detect characteristics of the patient's urine output (e.g., an amount and/or rate of urine output, and/or electrical, chemical, and/or physical properties of the patient's urine including, e.g., urine sodium concentration, urine conductivity, urine temperature, urine oxygen content, etc.). As described in detail below (e.g., with reference to FIGS. 3 and 4), the sensor(s) 114 can be integrated into a urine flow analysis cartridge 101 (“cartridge 101”) configured to receive urine from the patient P. The cartridge 101 can include a flow channel fluidly coupled to the fluid line 119, and the sensor(s) 114 can be positioned within the flow channel. Accordingly, as urine flows through the cartridge 101, the sensor(s) 114 can generate data based at least partially on the patient's urine such that the controller 140 can monitor and/or compute the patient's urine output based on the data generated by the sensor(s) 114. In some embodiments, the fluid line 119 can be coupled directed to the cartridge 101, for example, to a fluid inlet of the cartridge 101. Additionally or alternatively, the cartridge 101 can be integrated with the console 105, detachably coupled to the console 105, integrated into the fluid line 119, and/or have another suitable configuration relative to one or more other components of the system 100.

The urine output can be determined in many different ways, such as based on urine flow (e.g., through the fluid line 119, the cartridge 101, and/or into the container 112), the amount of urine in the container 112 (e.g., based on the weight of the container 112, level of urine in the container 112, etc.), and/or other properties associated with the urine. The sensor(s) 114 can include one or more of the following: a flow sensor, drip counter, fluid weight sensor, fluid level sensor, float sensor, optical sensor, ultrasonic sensor, and/or other sensors known in the art suitable for measuring a urine output amount and/or rate. In the embodiment of FIG. 1, the sensor(s) 114 are positioned at the console 105. In other embodiments, however, some or all of the sensor(s) 114 can be at a different location in the system 100, such as on or in the line 119, on or in the container 112, and/or on or in the patient P.

In some embodiments, the sensor(s) 114 can include at least one sensor configured to measure one or more characteristics of the urine, in addition to detecting the patient's urine output. For example, the sensor(s) 114 can be configured to measure urine temperature, urine conductivity, urine oxygenation, urine specific gravity, and/or levels of one or more analytes in the urine (e.g., creatinine, sodium, potassium, etc.). Such characteristics can be useful, e.g., in determining effectiveness of a particular therapy and/or whether the patient P is in or could be approaching a critical condition. For example, urine conductivity and/or urine electrolytes (e.g., sodium) can indicate whether the patient is responding well to the fluid therapy, or whether the patient is in a critical condition and fluid therapy should cease. In some embodiments, urine conductivity (either alone or in combination with urine specific gravity) is used as a proxy for measurements of urine sodium and/or other urine electrolytes, e.g., a higher urine conductivity can correlate to higher urine sodium levels and a lower urine conductivity can correlate to lower urine sodium levels. As another example, urine temperature measurements can be used to detect urine flow (e.g., based on heat loss through the fluid line 119). The urine temperature can also be used as a proxy for the patient's body temperature, which in turn can correlate to the patient's current clinical state.

Optionally, the sensor(s) 114 can include at least one sensor configured to monitor the status of the urine collection procedure, such as whether urine collection is proceeding normally, whether there are interruptions in urine flow, whether there is a blockage or leak in the urine system 110, the cartridge 101, etc. For example, the sensor(s) 114 can include a leak sensor configured to detect whether a leakage is present in the urine system 110 (e.g., at or near the fluid line 119, catheter 118, cartridge 101, and/or container 112). Leaks can be detected based on changes in urine flow rate, changes in pressure, the presence of moisture, or any other suitable parameter. In some embodiments, the controller 140 is configured to analyze the data from the leak sensor and/or other sensor(s) 114 to differentiate between low urine output rates versus leaks in the urine system 110.

As another example, the sensor(s) 114 can include a pressure sensor configured to measure the fluid pressure in the fluid line 119. The controller 140 can use the pressure measurements to monitor the status of urine flow, and optionally, detect whether there are any interruptions (e.g., decreases, sudden stoppages) or other issues with urine collection. In some embodiments, the controller 140 analyzes the pressure measurements to determine whether interruptions are due to low urine flow (e.g., the patient's bladder is empty or nearly empty), an air lock or other obstruction in the fluid line 119, a leak in the urine system 110 and/or a kink in the fluid line 119 and/or catheter 118. The controller 140 can alert the user if manual intervention is helpful or needed (e.g., to clear the obstruction, fix the leak, remove kinks from the fluid line 119, etc.). In embodiments where the urine system 110 includes a pump, the controller 140 can automatically activate the pump and/or increase the pumping rate to clear the obstruction from the fluid line 119.

The hydration system 120 can include at least one hydration fluid source 122 (“fluid source 122”—a bag, bottle, reservoir, etc.) containing a hydration fluid, such as saline (e.g., a premixed saline solution), Ringer's lactate solution, and/or other any other liquid solution suitable for infusion in the patient P to prevent or treat dehydration. The hydration fluid can be isotonic, hypertonic, or hypotonic, e.g., depending on the patient's condition and/or other treatment considerations. Optionally, the composition of the hydration fluid (e.g., sodium, chloride, potassium, bicarbonate, etc.) can be varied based on the patient's condition and/or expected or measured electrolyte loss during the treatment procedure.

The fluid source 122 can be connected to the patient P via at least one fluid line (e.g., an IV line or other tubing), such as first fluid line 129a and a second fluid line 129b. The fluid source 122 can be operably coupled to one or more hydration fluid components 124 for actuating and/or monitoring hydration fluid infusion via the first and second fluid lines 129a-b, such as a hydration fluid pump 126 and/or at least one hydration fluid sensor 128 (“fluid sensor 128”). In the illustrated embodiment, the fluid source 122 is fluidly coupled to the hydration fluid pump 126 via the first fluid line 129a, and the hydration fluid pump 126 can pump the hydration fluid into the patient P via the second fluid line 129b. The hydration fluid pump 126 can be or include a peristaltic pump or other pump suitable for infusing a fluid into the patient's body (e.g., via an IV route or another route).

The fluid sensor 128 can be configured to determine an amount and/or rate of hydration fluid flowing from the fluid source 122 toward the patient P, and can include a flow sensor, pressure sensor, and/or other sensor configured to determine fluid output from the pump 126. Alternatively, or in combination, the fluid sensor 128 can monitor hydration infusion rate by measuring the pumping rate of the pump 126 (e.g., the number of rotations of the pump 126 per minute). As described elsewhere herein, the controller 140 can be operatively coupled to the hydration system 120 and can receive sensor data from the fluid sensor 128 to determine a hydration fluid infusion rate. The controller 140 can control the pumping rate of the pump 126 to control the amount and/or rate of hydration fluid provided to the patient P.

Optionally, the amount of hydration fluid in the fluid source 122 can be monitored, e.g., based on weight, volume, fluid levels, flow rates, etc. In such embodiments, the fluid source 122 can be operably coupled to an additional sensor separate from the fluid sensor 128 (not shown), such as a fluid level monitor, float sensor, weight sensor, optical sensor, drip counter, flow measurement sensor, or the like. The additional sensor can provide an independent source of measurement data for determining and/or verifying the amount and/or rate of hydration fluid being provided to the patient P, which can be helpful for improving measurement accuracy.

In some embodiments, the hydration system 120 includes at least one sensor configured to detect the presence of the fluid source 122, such as a location sensor, optical sensor, weight sensor, etc. The hydration system 120 can use the sensor data to automatically determine whether the fluid source 122 is present or absent, e.g., to assess whether the system 100 is ready to initiate the fluid therapy treatment. Optionally, the sensor data can be used to detect if the user is removing the fluid source 122 during the treatment procedure, e.g., to switch an empty or nearly empty fluid source 122 with a new fluid source 122. In such embodiments, the system 100 can automatically pause hydration fluid infusion until the fluid source 122 has been replaced. Accordingly, the user can switch fluid sources 122 without having to inform the system 100 or manually pause the procedure.

The diuretic system 130 can be configured to automatically provide a diuretic to the patient P. The diuretic system 130 can include a diuretic source 134 (e.g., syringe, bag, reservoir, etc.) containing a diuretic, such as bumetanide (Bumex®), ethacrynic acid (Edecrin®), furosemide (Lasix®), torsemide (Demadex®), and/or other diuretics known in the art, each of which may be part of a fluid solution (e.g., a mixture of saline and a diuretic or other agent). In some embodiments, the identity and/or concentration of the diuretic can be received by the controller 140 via user input (e.g., using the display 150), by scanning a barcode of the diuretic source 134 or other container of the diuretic, and/or any other suitable technique.

The diuretic source 134 can be connected to the patient P via a fluid line 139 (e.g., an IV line or other tubing). The diuretic source 134 can also be operably coupled to one or more diuretic components 136 for actuating and/or monitoring diuretic delivery via the fluid line 139. For example, the diuretic components 136 can include a diuretic pump configured to pump the diuretic through the fluid line 139 and toward the patient P. The diuretic pump can include a peristaltic pump, a syringe pump, a metering pump, or other device suitable for delivering the diuretic to the patient P at a plurality of dosage rates. The diuretic pump can deliver the diuretic according to any suitable delivery profile, such as at a controlled continuous rate and/or in controlled boluses delivered at regular intervals through the fluid line 139.

In some embodiments, the diuretic pump is or includes a syringe pump having a mechanical injector or plunger that is operably coupled to the controller 140, such that the controller 140 causes movement of the injector to transfer the diuretic to the patient P. The syringe pump can include or be coupled to an actuator that mechanically drives the injector to control the delivery of the diuretic to the patient P. For example, the actuator can be or include a mechanical actuator, such as a nut for rotating a screw to drive the injector. The syringe pump can also include or be operably coupled to a sensor for detecting the position of the injector. Alternatively, or in combination, the diuretic pump can include other types of pumps and/or actuators. For example, the diuretic pump can include a motor, a gearbox operatively connected to the motor, a sensor for measuring rotation of said motor (e.g., a tachometer or an optical encoder), and/or a microcontroller configured to control operation of the motor and monitor the quantity of diuretic delivered to the patient P. As another example, the diuretic pump can include an electric motor, such as a rotary motor, a linear motor, and/or a series of electrically actuated solenoids configured to propel liquid from the diuretic source 134 and through the line 139 toward the patient P.

In some embodiments, the diuretic components 136 include one or more diuretic sensors configured to determine an amount and/or rate of diuretic flowing toward the patient P. The one or more diuretic sensors can include, for example, a flow sensor, weight sensor, and/or other sensor type configured to determine the amount and/or rate of diuretic delivered from the diuretic source 134. Optionally, the diuretic sensors can measure diuretic delivery based on the output from the diuretic pump, such as by monitoring the pumping rate (e.g., number of rotations of the diuretic pump per minute, plunger position, etc.). The diuretic components 136 can include additional functional components, such as an air bubble detector, pressure sensor, extravasation sensor (e.g., ivWatch device), and/or other embedded electronics, e.g., to provide feedback signals to the controller 140 to ensure accurate diuretic infusion and/or monitor infusion status.

The controller 140 is configured to automatically control hydration fluid and/or diuretic infusion (e.g., based at least in part on the patient's urine output) to promote safe and effective diuresis of the patient P. The controller 140 can include one or more processor(s) and tangible, non-transient memory configured to store programmable instructions. The controller 140 can be operably coupled to the urine system 110, hydration system 120 and/or diuretic system 130 to receive data (e.g., sensor data) from and transmit data (e.g., control signals) to the various components of these systems. For example, the controller 140 can receive sensor data from the urine system 110 (e.g., from sensor(s) 114) to determine and/or monitor the patient's urine output. Based on the urine output, the controller 140 can determine an appropriate diuretic dosage amount and/or rate to administer to the patient P, and can cause the diuretic system 130 to deliver the diuretic accordingly. For example, the controller 140 can determine a pumping rate of the diuretic pump to produce the desired delivery profile for the diuretic. Similarly, the controller 140 can determine an appropriate hydration fluid infusion rate for the patient P (e.g., based on the urine output and/or the diuretic dosage rate), and can cause the hydration system 120 to deliver the appropriate hydration fluid amount and/or rate. For example, the controller 140 can determine a pumping rate for the hydration fluid pump 126 to achieve the desired hydration fluid infusion rate. The controller 140 can regulate the diuretic dosage rate and/or hydration fluid infusion rates based on a suitable treatment regimen protocol, e.g., prescribed by a physician and/or managed by the controller 140.

During the procedure, the controller 140 can receive sensor data from the various sensors of the urine system 110, hydration system 120 and/or diuretic system 130 to monitor the urine output, hydration fluid infusion rate, and/or diuretic dosage rate, respectively. The controller 140 can also receive sensor data from additional sensors configured to monitor patient status and/or operational status of the system 100, such as fluid pressure sensors, blood pressure sensors, air bubble detectors, analyte detectors, and the like. For example, the controller 140 can be operably coupled to at least one sensor implanted in, attached to, or otherwise associated with the patient P. The sensor(s) can provide data regarding any of the following patient parameters: pressure levels (e.g., pulmonary artery pressure, left atrial pressure), bioelectric measurements (e.g., bioimpedance vector analysis (BIVA)), hemoglobin measurements (e.g., non-invasive hemoglobin measurements), urine oxygenation levels, urine composition (e.g., creatine, sodium, potassium, chloride, etc.), urine temperature, body temperature (e.g., bladder temperature), oral fluid intake, heart rate, heart rate variability, blood oxygenation, hematocrit, hemodynamic data, and any other data described herein. The controller 140 can use the data from any of the sensors described herein to monitor treatment progress (e.g., whether the treatment is complete), patient status (e.g., whether the patient is responding well or poorly to treatment), and/or potential safety concerns (e.g., whether the diuresis is too aggressive, whether the patient is exhibiting side effects). The controller 140 can also adjust the hydration fluid infusion rate and/or diuretic dosage rate based on the sensor data. Additionally, the sensor data can also provide feedback to the controller 140 to confirm or verify the effectiveness of the fluid therapy.

The controller 140 can also use other data for monitoring and/or controlling the therapy, such as settings for the system 100, user input, data indicative of a desired treatment regimen (e.g., a programmed diuretic and/or hydration fluid delivery profile over time), and/or other data collected or calculated by the controller 140. In some embodiments, the data used by the controller 140 includes current and/or historical data for the patient P, such as diuretic dosages delivered to the patient P, urine output volume or rate, the amount of hydration fluid infused into the patient P, the weight or change in weight of the patient P at various times during the infusion of the diuretic, indicators of the patient's renal function (e.g., estimated glomerular Filtration Rate (eGFR)), and/or the time(s) during which the patient P was treated with the system 100. Additionally or alternatively, the data used by the controller 140 can include historical data for one or more other patients, as described elsewhere herein.

The display 150 (e.g., a touchscreen, monitor, etc.) can include a user interface configured to receive inputs from the user and display outputs to the user. In some embodiments, the display 150 is operatively coupled to the controller 140 and thus can be used to receive user input indicating treatment parameters, such as parameters for urine output, hydration fluid infusion, and/or diuretic dosage. The treatment parameters can include, for example: a desired fluid balance level (e.g., a positive, negative, or neutral fluid balance), estimated excess fluid volume, target fluid removal volume (e.g., minimum and/or maximum amount of fluid to be removed), desired urine output level (e.g., a total amount of urine output; a target maximum, minimum, and/or average urine output rate), treatment duration (e.g., maximum and/or minimum duration of the treatment procedure; planned duration of the input balance level and/or urine output level), hydration fluid type, hydration fluid infusion rate (e.g., maximum, minimum, and/or average infusion rate), hydration fluid infusion profile (e.g., a function indicating how the amount and/or rate of hydration fluid infusion should vary over time), time limits associated with hydration fluid infusion (e.g., maximum and/or minimum time period for hydration fluid infusion), diuretic type, diuretic dosage (e.g., maximum and/or minimum dosage), diuretic dosage rate (e.g., maximum, minimum, and/or average dosage rate), diuretic dosage profile (e.g., a function indicating how the dosage amount and/or dosage rate of diuretic should vary over time), time limits associated with diuretic delivery (e.g., maximum and/or minimum time period for diuretic delivery), other fluids received by the patient during the procedure (e.g., volume of ingested fluid, volume of fluid from other medical agents besides the diuretic and/or hydration fluid), and/or suitable combinations thereof. Other patient-related inputs may also be received at the display 150 and can include, for example, the patient's sex, weight (e.g., “dry” weight), age, ethnicity, clinical state (e.g., renal function parameters, electrolyte levels such as serum chloride levels and/or urine sodium levels), medical history (e.g., outcomes of previous fluid removal procedures, prior response to fluid therapy, etc.), diagnoses (e.g., ADHF, CHF), medications (e.g., whether the patient is diuretic-naïve or diuretic-resistant), dietary factors (e.g., whether the patient is consuming a high-salt or low-salt diet, amount of oral fluid intake), etc.

Alternatively, or in combination, the user input via the display 150 can prompt the controller 140 to retrieve treatment parameters (e.g., maximum diuretic dosage, maximum continuous diuretic dosage, and minimum desired urine rate) from tables and/or other data sources. The data sources can be stored in the system 100 (e.g., in a memory associated with the controller 140) and/or can be stored in a separate device (e.g., a remote computing device). In some embodiments, the controller 140 retrieves data from a remote database and/or server via a communication network (e.g., a wired network, a wireless network, a cloud-based network, the Internet, and/or suitable combinations thereof). In such embodiments, the controller 140 can be operably coupled to a communication device and/or interface configured to transmit and receive data via the communication network.

The controller 140 can output the treatment parameters to the user via the display 150 for review and/or feedback. For example, the display 150 can show recommended treatment parameters for the patient P, such as recommendations for the diuretic, diuretic dosage rate (e.g., initial, maximum, and/or minimum dosage rate), hydration fluid infusion rate (e.g., initial, maximum, and/or minimum infusion rate), urine output rate (e.g., maximum and/or minimum output rate), treatment duration (e.g., maximum time period for diuretic and/or hydration fluid infusion; maximum total treatment duration), treatment escalation (e.g., thiazide, temporarily increased fluid matching, additional loop diuretic), end of treatment, and so on. As another example, the display 150 can output one or more predetermined treatment programs so the user can select the appropriate program for the particular patient P. Optionally, the user can modify any of the displayed treatment parameters, if desired.

During the treatment procedure, the controller 140 can output information regarding procedure status to the user via the display 150. For example, the controller 140 can display information regarding any of the following: urine output (e.g., current urine output rate and/or amount, urine output rate and/or amount over time, total amount of urine output so far), hydration fluid infusion (e.g., current infusion rate and/or amount, infusion rate and/or amount over time, total amount of hydration fluid infused so far), diuretic delivery (e.g., current dosage rate and/or amount, dosage rate and/or amount over time, total amount of diuretic delivered so far), fluid balance (e.g., current fluid balance, fluid balance over time, net fluid removal so far), system status (e.g., amount of hydration fluid remaining in the fluid source 122, amount of diuretic remaining in the diuretic source 134, remaining storage capacity in the container 112), treatment time (e.g., treatment start time, projected and/or planned treatment end time, total treatment duration so far), notifications (e.g., alerts, alarms, messages, recommendations, predictions, error messages), and the like. The user can review the displayed information, and, if appropriate, provide input instructing the controller 140 to adjust, pause, and/or stop the treatment procedure.

In some embodiments, the system 100 includes redundancy in the urine system 110, hydration system 120, and/or diuretic system 130 to reduce or minimize treatment interruptions, e.g., due to running out of urine collection capacity, running out of hydration fluid, and/or running out of diuretic. For example, the system 100 can include redundant components (e.g., containers 112, fluid sources 122, and/or diuretic sources 134), which can be stored at predetermined locations (e.g., on or within the console 105 or another portion of the system 100). The controller 140 can be configured to detect the presence of the redundant components, and can automatically or semi-automatically switch between these components so the treatment procedure can continue uninterrupted or substantially uninterrupted. Alternatively, or in combination, the system 100 can adjust the timing of user alerts related to urine collection capacity, hydration fluid levels, and/or diuretic levels, based on the availability of redundant components. For example, if redundant components are available, the system 100 can generate alerts at a later time (e.g., closer in time to when the container 112 would be full, when the fluid source 122 would be empty, and/or when the diuretic source 134 would be empty), since the system 100 can automatically switch to using the redundant components, or the user can rapidly perform the switch using the redundant components that are already stored locally at the system 100, rather than having to retrieve replacements from another location.

The lack of interruption in fluid therapy can help ensure effectiveness of the fluid therapy, e.g., by relieving the patient's fluid overload condition as quickly and safely as possible.

In some embodiments, even brief interruptions in diuretic delivery and/or hydration fluid infusion can significantly affect the patient's urine output (e.g., cause the urine output rate to drop), which can interfere with therapeutic efficacy and prolong treatment time. The concerns described above regarding diuretic and/or hydration fluid backup supply may be unique to the present technology, e.g., due to the relatively large amounts of diuretic and/or hydration fluid that are utilized over time in some embodiments of the treatment procedures described herein. That is, whereas conventional systems and methods may utilize just a single diuretic source and/or a single hydration fluid source because of the relatively low amount of diuretic and/or hydration fluid administered, the present technology may benefit from multiple diuretic sources and/or hydration fluid sources to ensure treatment continuity. Similarly, the treatment procedures of the present technology can cause the patient P to produce relatively large volumes and/or rates of urine output compared to conventional procedures, such that multiple containers 112 may be helpful to reduce the number of times the user has to empty and/or replace the containers 112 during the procedure.

For example, in some embodiments, the urine system 110 includes two or more redundant containers 112 to ensure fluid therapy does not need to be stopped or interrupted due to the container 112 being full. In such embodiments, the urine system 110 can include a flow control assembly 116 (e.g., valves and/or other flow control components) operably coupled to the controller 140, and configured to selectively direct the urine from the patient P to one or more of the containers 112. The flow control assembly 116 can initially direct the urine received from the patient P to a first container 112. Once the flow control assembly 116 detects or determines the first container is full or nearly full (e.g., based on sensor data from the sensor(s) 114), the flow control assembly 116 can redirect the urine received from the patient P to a second container 112. While urine is being directed to the second container 112, a user can empty the first container 112 or replace the first container 112 with an empty container 112. The flow control assembly 116 and/or controller 140 can generate an alert to the user to indicate the first container is full and needs to be replaced or emptied. This process can be repeated such that fluid management therapy is not inadvertently interrupted due to the containers 112 being full and/or the urine system 110 being unable to accept urine output. In some embodiments, the treatment procedures described herein result in relatively large amounts and/or rates of urine output (e.g., compared to conventional therapies), such that automatic switching between multiple urine containers is advantageous to minimize treatment interruptions.

As another example, the hydration system 120 can include multiple redundant hydration fluid sources 122, e.g., to ensure the hydration fluid infusion can continue without interruption for the entirety of a therapy session and/or to provide an additional time window for switching hydration fluid sources 122 without interrupting hydration fluid infusion. In such embodiments, the hydration system 120 can include a hydration control assembly (e.g., valves and/or other flow control components—not shown) operably coupled to the controller 140, and configured to switch the source of hydration fluid from a first fluid source 122 to a second fluid source 122. In such embodiments, the hydration control assembly can initially deliver hydration fluid from the first fluid source 122 to the patient P. The hydration control assembly can monitor whether the first fluid source 122 is empty or nearly empty, e.g., based on data from the fluid sensor 128 and/or other sensors associated with the hydration system 120. Once the hydration control assembly detects or determines the first fluid source 122 is empty or nearly empty (e.g., the remaining amount of hydration fluid is below a predetermined threshold), the hydration control assembly can switch to delivering hydration fluid from the second source 122. The switching process can be repeated such that fluid therapy is not inadvertently interrupted due to the fluid source 122 being empty and/or the hydration system 120 being unable to provide hydration fluid.

The process of switching the hydration fluid source 122 can be performed automatically, semi-automatically, or manually. In some embodiments, semi-automatic or manual switching between the first and second fluid sources 122 may be beneficial to ensure the hydration system 120 does not automatically infuse hydration fluid without user confirmation. In such embodiments, the hydration control assembly and/or controller 140 can output an alert asking the user to verify that the hydration fluid should be switched from the first fluid source 122 to the second fluid source 122. Upon switching to the second fluid source 122, the controller 140 can generate an alert to the user to indicate the first fluid source 122 is empty and needs to be replaced. Optionally, the hydration control assembly and/or controller 140 can implement a pre-approval procedure in which the user allows the hydration system 120 to automatically infuse a specified volume of additional hydration fluid. Once that volume has been delivered to the patient P, the user may need to provide re-approval before further automatic infusion of hydration fluid.

In some embodiments, the different fluid sources 122 of the hydration system 120 each provide the same type of hydration fluid. In other embodiments, however, some or all of the fluid sources 122 can provide different types of hydration fluid. The hydration fluids can differ from each other with respect to tonicity, composition, electrolyte content, etc. Depending on the patient's response to diuresis, the hydration system 120 can deliver multiple different hydration fluids to the patient P sequentially or concurrently. For example, if the patient's urine output indicates that the patient P has an electrolyte imbalance (e.g., a positive sodium balance), the hydration system 120 can switch to delivering a hydration fluid that would address the imbalance (e.g., a hydration fluid with lower sodium content). The switching can be performed using any of the techniques and/or devices described above. Accordingly, the particular fluid or fluids delivered to the patient P can be tailored to the patient's particular clinical state and/or response to treatment.

In yet another example, the diuretic system 130 can include multiple redundant diuretic sources 134, e.g., to ensure the diuretic delivery can continue without interruption for the entirety of a therapy session and/or to provide an additional time window for switching diuretic sources 134 without interrupting diuretic delivery. For example, if a first diuretic source 134 (e.g., a first syringe or container) is spent, the diuretic can continue to be supplied (e.g., without substantial interruption) via a second diuretic source 134 (e.g., a second syringe or container). The second diuretic source 134 can be connected to the console 105, and can be operably coupled to a sensor configured to detect the presence of the second diuretic source 134 (e.g., a location sensor, optical sensor, weight sensor, etc.). Accordingly, the diuretic system 130 can switch to the second diuretic source 134 if the first diuretic source 134 is empty or nearly empty, and the second diuretic source 134 is present.

In some embodiments, the diuretic system 130 includes two independent diuretic pumps each including its own diuretic source 134. For example, the diuretic system 130 can include syringe pumps each fluidly coupled to its own syringe filled with diuretic. In some cases, such syringes may only be filled by pharmacists or other health care professionals, and thus may not be readily replaced (e.g., in less than a few hours) by the user. When the diuretic system 130 and/or controller 140 detects that the first diuretic source 134 is empty or nearly empty (e.g., below a predetermined threshold), the diuretic supply can be switched (e.g., automatically or manually) to a second diuretic source 134. The switching process can include stopping a first syringe pump fluidly coupled to the first syringe, and starting a second syringe pump fluidly coupled to the second syringe. In other embodiments, the diuretic system 130 includes a single diuretic pump (e.g., syringe pump) connected to two diuretic sources 134. In such embodiments, case switching between the first and second diuretic sources 134 can involve using a diuretic control assembly (e.g., valves and/or other flow control components) to switch the diuretic pump from delivering diuretic from the first diuretic source 134 to the second diuretic source 134. The switching process can be repeated such that fluid therapy is not inadvertently interrupted due to the diuretic source 134 being empty and/or the diuretic system 130 being unable to provide diuretic.

The process of switching the diuretic source 134 can be performed automatically, semi-automatically, or manually. In some embodiments, manual or semi-automatic switching between the first and second diuretic sources 134 may be beneficial to ensure the diuretic system 130 does not automatically infuse a large volume of diuretic without user confirmation. In such embodiments, the controller 140 can output an alert asking the user to verify that the diuretic should be switched from the first diuretic source 134 to the second diuretic source 134. Upon switching to the second diuretic source 134, the controller 140 can generate an alert to the user to indicate the first diuretic source 134 is empty and needs to be replaced. Optionally, the controller 140 can predict a time point and/or time range when the first diuretic source 134 will be empty (e.g., based on the diuretic dosage rate), and can output a notification so the user can order or otherwise prepare a replacement diuretic source 134 before the first diuretic source 134 runs out. Moreover, the diuretic control assembly and/or controller 140 can implement a pre-approval procedure in which the user allows the diuretic system 130 to automatically delivery a specified additional dosage of diuretic. Once that dosage has been delivered to the patient P, the user may need to provide re-approval before further automatic delivery of diuretic.

In some embodiments, the different diuretic sources 134 of the diuretic system 130 each provide the same type of diuretic. In other embodiments, however, some or all of the diuretic sources 134 can provide different types of diuretics. Depending on the patient's response to diuresis, the diuretic system 130 can deliver multiple different diuretics to the patient P sequentially or concurrently. For example, the diuretic system 130 can initially deliver a first diuretic to the patient P from a first diuretic source 134. If the patient P responds poorly to the first diuretic (e.g., the urine output rate does not increase or increases very slowly), the diuretic system 130 can switch to delivering a second, different diuretic from a second diuretic source 134. The diuretic system 130 can continue delivering the first diuretic concurrently with the second diuretic, or can terminate delivery of the first diuretic when the second diuretic is delivered. The switching can be performed using any of the techniques and/or devices described above. As another example, if the patient P does not respond well to a single diuretic, the diuretic system 130 can simultaneously administer multiple diuretics to the patient P. The ratio of the different diuretics can be varied as appropriate to elicit a suitable urine output rate. In other embodiments, however, rather than automatically administering additional diuretics, the diuretic system 130 can output a notification recommending that the user manually administer a different diuretic to the patient P and/or requesting that the user approve administration of a different diuretic, which may be beneficial for patient safety.

The system 100 illustrated in FIG. 1 can be configured in many different ways. For example, the locations of the various components of the system 100 can be altered, e.g., the urine system 110, hydration system 120, and/or diuretic system 130 can be at different locations in the console 105. As another example, any one of the urine system 110, hydration system 120, or diuretic system 130 can be part of a separate system or device (e.g., a separate console), or can be omitted altogether. For instance, in some embodiments, the urine system 110 is replaced with a mechanism for monitoring the patient's urine output that does not require the catheter 118 and/or urine collection, such as an ultrasound sensor that measures the patient's bladder volume. The ultrasound sensor can be implemented as a patch or similar device that is coupled to the patient's body. The controller 140 can process the ultrasound sensor data to detect changes in the bladder volume, and can determine the corresponding amount and/or rate of urine output based on the bladder volume. The use of non-invasive urine monitoring mechanisms such as an ultrasound sensor can allow the treatment procedures described herein to be performed in outpatient settings.

As another example, in some embodiments, the hydration system 120 is omitted such that diuresis is performed without hydration fluid infusion, or the hydration fluid is infused manually. Diuresis with hydration fluid infusion may be more beneficial for patients with low serum chloride levels (e.g., patients with low-salt diets), while patient with high serum chloride levels (e.g., patients with high-salt diets) may tolerate diuresis with little or no hydration fluid infusion. Optionally, the hydration fluid infusion rate can be varied at least partially based on the patient's serum chloride levels, e.g., lower amounts and/or rates of hydration fluid infusion can be used if the patient's serum chloride level is high (e.g., greater than or equal to 105 mmol/L).

In yet another example, the diuretic system 130 can be omitted such that no diuretic is administered, or the diuretic is administered manually. In such embodiments, the system 100 can provide automated fluid replacement via the hydration system 120 and/or can automatically monitor the patient's urine output via the urine system 110, but the diuretic would be administered manually by a healthcare professional in accordance with techniques known to those of skill in the art.

The system 100 can optionally include or be used in combination with additional systems or devices, such as systems or devices configured to perform any the following functions: administering other medications and/or agents besides the diuretic and hydration fluid (e.g., heart failure medication), monitoring other patient parameters besides urine output (e.g., blood pressure, weight, heart rate, blood oxygenation, respiratory rate, temperature), and/or performing other types of medical procedures on the patient P concurrently or sequentially with the fluid removal procedure (e.g., dialysis, ultrafiltration).

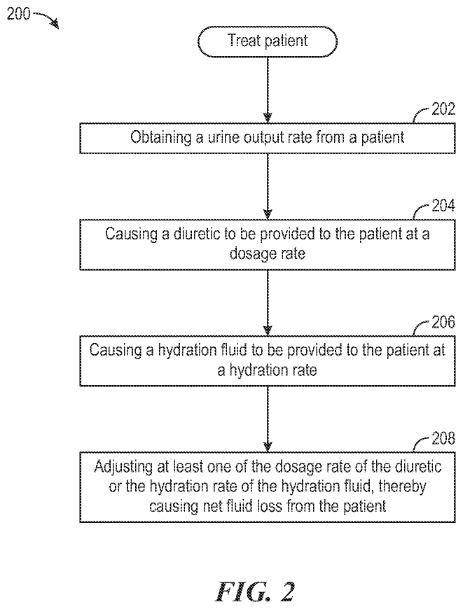

FIG. 2 is a flow diagram of a method 200 for treating a patient, in accordance with embodiments of the present technology. In some embodiments, the method 200 is used to treat the patient for fluid overload by removing fluid from the patient to produce a negative fluid balance (net fluid loss). The method 200 can be performed by any embodiment of the systems and devices described herein, such as the system 100 of FIG. 1. In some embodiments, some or all of the blocks of the method 200 are performed by a system or device including one or more processors and a memory storing instructions that, when executed by the one or more processors, cause the system or device to perform one or more of the blocks described herein. For example, the method 200 can be performed by the controller 140 of the system 100 of FIG. 1 and/or another suitable processor. Optionally, some or all of the blocks of the method 200 can performed automatically or semi-automatically, with little or no human intervention.

The method 200 can begin at block 202 with obtaining a urine output rate from a patient. The urine output rate can be obtained from a urine monitoring and/or collection system connected to the patient, such as the urine system 110 of FIG. 1. The system can determine the urine output rate based on received input data, such as data from one or more sensors (e.g., the sensor(s) 114 of FIG. 1). As described above, the sensor(s) can be configured to measure the urine output rate based on flow rate, weight (e.g., of the container 112 of FIG. 1), volume, fluid level, and/or any other suitable parameter. The urine output rate can be calculated based on the received input, e.g., by a controller (e.g., controller 140 of FIG. 1) operatively coupled to the sensor(s). The urine output rate can be a current rate or an average rate measured over a predetermined time period (e.g., the previous 5 or 10 minutes). The urine output rate can be updated on a continuous or recurring basis (e.g., every 30 seconds, 1 minutes, 2 minutes, etc.). In some embodiments, the process of block 202 is performed concurrently with some or all of the other blocks of the method 200 (e.g., blocks 204, 206, and/or 208) to provide continuous or substantially continuous urine output monitoring through the entirety of the method 200.

At block 204, the method 200 optionally continues with causing a diuretic to be provided to the patient at a dosage rate. The diuretic can be or include furosemide, bumetanide, ethacrynic acid, torsemide, combinations thereof, and/or other diuretics known in the art. In some embodiment, the diuretic is delivered as part of a solution including saline or other hydration fluid(s) mixed therewith. The diuretic can be provided automatically or semi-automatically by a diuretic system connected to the patient, such as the diuretic system 130 of FIG. 1. The diuretic system can be operably coupled to a controller (e.g., controller 140 of FIG. 1) for causing diuretic delivery in accordance with a planned and/or pre-programmed treatment procedure.

In some embodiments, the treatment procedure includes multiple phases, and each phase is associated with a different delivery profile for the diuretic. In such embodiments, block 204 can be performed as part of an initial phase to determine an appropriate diuretic dosage rate for treating the patient (also known as a “dosage determining phase”). In the dosage determining phase, the diuretic is injected at an initial dosage rate, and the dosage rate can then be gradually increased (e.g., “ramped”) to elicit an increase in the patient's urine output rate. The diuretic dosage rate can be increased according to a desired function or delivery profile, such as a continuous function, a block-wise function, or a combination thereof). The function can include iteratively increasing the dosage rate linearly, exponentially, according to a polynomial function, and/or any other suitable ramp function or profile. In some embodiments, the diuretic is delivered in a manner such that a subsequent dosage rate is a predetermined percentage (e.g., at least 5%, 10%, 15%, 25%, etc.) above the immediately previous dosage rate. The predetermined percentage can increase or decrease over time, e.g., depending on the desired fluid therapy and/or patient considerations. Optionally, the diuretic can be provided in a manner that doubles the diuretic dosage rate or total diuretic within a period of time (e.g., 10 minutes, 15 minutes, 20 minutes, or within a range of 10-20 minutes). In other embodiments, however, the dosage determining phase can include one or more time periods during which the diuretic dosage rate does not increase and/or is held substantially constant. The dosage determining phase can continue until the patient's urine output reaches or exceeds a desired threshold rate and/or a predetermined time period has elapsed, at which point the diuretic dosage rate can be adjusted, as described in block 208 below.

At block 206, the method 200 can optionally include causing a hydration fluid to be provided to the patient at a hydration rate. The hydration fluid can comprise saline and/or other fluids having sodium, and can be provided automatically or semi-automatically by a hydration fluid system connected to the patient, such as the hydration system 120 of FIG. 1. The hydration fluid can be provided before, during, and/or after providing the diuretic in block 204 (e.g., before, during, and/or after the dosage determining phase). Intravenous infusion of hydration fluid containing electrolytes (e.g., sodium and/or chloride) can increase diuretic efficiency, which is counterintuitive since a goal of fluid therapy is net removal of fluid. Hydration fluid can also reduce or inhibit intravascular depletion, decreases in cardiac output, and/or decreases in renal perfusion, among other benefits.

In some embodiments, the hydration fluid is provided to the patient based at least in part on the corresponding urine output rate, e.g., to drive net fluid loss from the patient. For example, the hydration rate can be less than the urine output rate. In some embodiments, the hydration rate is a percentage of the urine output rate (e.g., 90%, 80%, 70%, 60%, 50%, 40%, 30%, 20%, or 10% of the urine output rate) for a given range of urine output rates (e.g., from 0 mL/hr to 1000 mL/hr). Optionally, the percentage can be higher for certain parts of the range (e.g., for the lower end of the range to reduce the likelihood of hypotension) and/or lower for other parts of the range (e.g., for the higher end of the range to increase net fluid loss). As another example, the hydration rate can substantially match the urine output rate (e.g., 100% of the urine output rate) for an initial amount of urine output by the patient (e.g., at least the initial 150 mL, 200 mL, or 250 mL), for an initial time period (e.g., the first hour, 2 hours, or 3 hours), for an initial time period during hydration fluid and/or diuretic dose finding, and/or until the patient's urine output rate reaches a predetermined threshold. Subsequently, the hydration rate can be adjusted to be less than the urine output rate. In a further example, the hydration rate may be determined based on whether the urine output rate is above or below one or more different thresholds, with the difference between the urine output rate and hydration fluid rate increasing as the urine output rate increases. In such embodiments, the difference between the urine output rate and the hydration fluid rate can increase (with the urine rate being higher than the hydration fluid rate) as the urine output rate increases, and thus the net fluid loss from the patient can increase as the urine output rate increases.

At block 208, the method 200 can include adjusting at least one of the dosage rate of the diuretic or the hydration rate of the hydration fluid, thereby causing net fluid loss from the patient. For example, the (i) diuretic dosage rate can be adjusted, (ii) the hydration rate can be adjusted, or (iii) the diuretic dosage rate and the hydration rate can both be adjusted. In some embodiments, the diuretic dosage rate is adjusted after the dosage determining phase of the treatment procedure is complete. As discussed above in block 204, the dosage determining phase can end when (i) a predetermined amount of time has elapsed since the initial diuretic administration, and/or (ii) the urine output rate is or becomes greater than or equal to a predetermined threshold rate. The treatment procedure can then switch to a phase in which the diuretic dosage rate is adjusted to a dosage rate configured to maintain the patient's urine output rate at or above a desired output rate to cause net fluid loss (also known as a “continuous delivery phase” or “fluid reduction phase”).

The adjusted diuretic dosage rate can be the initial dosage rate for the fluid reduction phase, and can be determined in many different ways. For example, the adjusted diuretic dosage rate can be based on the outcome of the dosage determining phase (e.g., the dosage rate when the patient's urine output reaches or exceeds the target threshold). In representative embodiments, the diuretic dosage rate is decreased, e.g., to maintain the patient's urine output rate at a predetermined rate and/or within a predetermined range (e.g., no more than 5%, 10%, or 20% variability from a predetermined rate). Decreasing the diuretic dosage rate can decrease the rate of increase in urine output rate (e.g., cause the patient's urine output to approach a constant or substantially constant rate) but without actually decreasing the urine output rate itself.

In some embodiments, the adjusted diuretic dosage rate is a predetermined percentage or fraction of the current dosage rate (e.g., the dosage rate at the end of the dosage determining phase) or a predetermined percentage of the cumulative diuretic dosage amount (e.g., the cumulative amount delivered during the dosage determining phase). For example, the adjusted dosage rate can be a predetermined percentage (e.g., 10%, 15%, 20%, 25%, 30%, or within a range of 10-30%) of a value of the total amount of diuretic delivered to the patient at that time. For example, if the total amount delivered is 100 mg, and the predetermined percentage is 25%, then the adjusted dosage rate can be 25 mg/hr. In some embodiments, the percentage used to calculate the adjusted diuretic dosage rate is based on a pharmacokinetic characteristic of the particular diuretic being infused. For example, the percentage can be 20% for furosemide, such that if 50 mg of furosemide is infused in 60 minutes, then the adjusted diuretic dosage rate can be 10 mg/hr.

In some embodiments, block 208 includes delivering the diuretic at the adjusted diuretic dosage rate until the fluid reduction phase is complete, e.g., until a predetermined period of time has elapsed, the patient's urine output drops below a low urine output threshold, and/or until a target net fluid loss volume is achieved. During the fluid reduction phase, the diuretic dosage rate can be constant or substantially constant (e.g., no more than 5%, 10%, or 20% variability from the initially determined adjusted diuretic dosage rate). In other embodiments, however, block 208 can include making additional adjustments to the diuretic dosage rate during the treatment procedure (e.g., increasing and/or decreasing the diuretic dosage rate). The adjustments can be based on whether one or more of a predetermined set of conditions is met, such as whether the urine output rate is too high (e.g., which can indicate that the patient has a high and/or increasing serum level of diuretic). The set of conditions can include (i) an average urine rate being greater than a predetermined rate for a period of time, (ii) an average rate of change of the urine rate being greater than a predetermined rate of change, and/or (iii) a diuretic dosage rate being greater than a predetermined dosage rate. If some (e.g., two) or all of the conditions are met, the diuretic dosage rate can be decreased (e.g., by a predetermined amount or percentage), also referred to herein as “down-titration.”

In some embodiments, a down-titration is performed only if all or a majority of the above conditions are met, which can avoid unnecessarily decreasing the diuretic dosage rate, thereby allowing urine output rates to remain high and avoiding unnecessary interruptions to the treatment procedure. For example, whereas other methodologies may interrupt fluid therapy and decrease the diuretic dosage rate (e.g., to zero mg/hr) only when the urine rate too high, the process described herein may only decrease the dosage rate (e.g., to a non-zero or zero dosage rate) when one or more factors are met, such as when the urine output rate is both high and continuing to increase. Stated differently, the process herein can prevent the diuretic dosage rate from being unnecessarily decreased when urine rates are temporarily high (e.g., above the predetermined rate), but are trending downward. This approach can prevent or inhibit over-diuresis, excess fluid loss and/or electrolyte loss, as well limit unnecessary exposure of the patient to additional diuretic. Additionally, because the diuretic dosage rate can be down-titrated, rather than stopping the diuretic entirely, the fluid therapy can continue (albeit at lower urine output rates) without needing to completely restart the procedure.

As another example, the additional adjustments to the diuretic dosage rate in block 208 can include increasing the diuretic dosage rate, also referred to herein as “re-ramping” or “up-titration.” In some embodiments, re-ramping is performed if urine output rates are too low, as determined based on a set of conditions. The set of conditions can include (i) the average urine rate being below a predetermined threshold rate for a predetermined period of time, and/or (ii) more than a predetermined amount of debt has accumulated over the predetermined period of time. “Debt” can be defined as the area on a plot between the urine output rate and a set rate (e.g., 325 mL/hr), and can represent how much of and for how long the urine output rate has been below the set rate. If some or all of the conditions are met, re-ramping can be performed by incrementally increasing the diuretic dosage rate until (i) a predetermined amount of time has elapsed, and/or (ii) the urine output rate is or becomes greater than or equal to a predetermined threshold rate. The re-ramp process can be identical or generally similar to the dosage determining process previously described in block 204. In representative embodiments, the dosage rate or “ramp” can start at any dosage identified during the dosage determining process, such as the current dosage rate, a previously determined dosage rate, or another suitable dosage rate (e.g., not at the beginning of the dosage).

The re-ramping process can be performed automatically, semi-automatically, or manually. In some embodiments, re-ramping is a semi-automatic or manual process requiring user approval, e.g., for regulatory and/or safety reasons. In such embodiments, the system can output a notification to the user (e.g., via the display 150 of FIG. 1) instructing the user to confirm that re-ramping should be initiated. Optionally, the system can implement a pre-approval procedure in which the user can allow the system to automatically perform re-ramping under certain conditions (e.g., within a specific time period, until a certain urine output volume and/or rate is achieved, for a maximum diuretic amount and/or dosage rate, etc.). This approach can allow for automatic re-ramping under limited circumstances, which can reduce the amount of human intervention during the treatment procedure and improve the responsiveness of the system to the patient's current state. Once the pre-approval conditions have elapsed, the user may need to provide re-approval before additional automatic re-ramping is allowed.

In some embodiments, block 208 also includes adjusting the diuretic dosage rate in response to a potential and/or detected blockage (e.g., an air lock, a kink in a fluid line, etc.) in the urine collection system. For example, an air lock can be any partial or complete obstruction of fluid flow due to trapped gas (e.g., air) within a fluid system. Air locks may produce an artificial drop in urine output rates, which can affect the determination of the diuretic dosage rate (e.g., result in a diuretic dosage rate that is too high). In some embodiments, the presence of an air lock is detected based on a period of little or no urine output (due to the air lock blocking urine flow), followed by a sudden large bolus of urine output (due to built-up pressure in the fluid line clearing the air lock). When the system detects that an air lock or other blockage was or is present, the system can compensate by adjusting the diuretic dosage rate to the dosage rate that should have been used if the air lock or other blockage had not occurred. The appropriate dosage rate can be determined based on historical data for the patient receiving the fluid therapy and/or one or more other patients (e.g., the diuretic dosage rate before the air lock occurred, a diuretic dosage rate calculated from the patient's urine output rate before the air lock occurred, etc.).

Alternatively, or in combination, block 208 can include adjusting the hydration rate, e.g., by increasing or decreasing the hydration rate based on the patient's urine output rate to drive net fluid loss from the patient. For example, as previously described, the hydration rate can initially match the patient's urine output rate for a set of initial conditions (e.g., certain time period, initial urine output amount, and/or initial urine output rate). Once the initial conditions have elapsed, the hydration rate can be maintained at a rate lower than the urine output rate (e.g., a percentage of the urine output rate) so the patient exhibits net fluid loss during the fluid reduction phase. The hydration rate can be determined in various ways, such as a percentage or fraction of the patient's urine output rate, based on whether the urine output rate is above or below a number of different thresholds (e.g., with the difference between the urine output rate and hydration rate increasing as the urine output rate increases), and/or any other suitable approach.

Optionally, the diuretic dosage rate and/or hydration rate can be adjusted based on factors other than patient's urine output rate. For example, in addition to or in lieu of the urine output rate, the diuretic dosage rate and/or hydration rate can be adjusted based on any one or combination of the patient's urine conductivity, urine sodium concentration, urine temperature, urine oxygen levels, etc. In some embodiments, the diuretic dosage rate and/or hydration rate can be adjusted based on the patient's blood pressure in order to avoid placing the patient in a hypotensive state. In some embodiments, if the patient's blood pressure level is too low (e.g., below a threshold value or range), the system can avoid increasing the diuretic dosage rate and/or can decrease the diuretic dosage rate for a certain period of time. Alternatively, or in combination, the system can increase the hydration rate (e.g., to the maximum allowable hydration rate and/or to provide a desired fluid replacement profile (e.g., a 100% match to the patient's urine output rate)) for a certain period of time if low blood pressure levels are detected. The system can also output an alert indicating that the patient's blood pressure level is low so a user can check on the patient's status. Optionally, the system can take both blood pressure levels and urine output rates into account, e.g., the system can generate alerts and/or can adjust the diuretic dosage rate and/or hydration rate if the patient's blood pressure is low and the patient's urine output rate drops. This approach can improve patient safety and control over the treatment procedure.

In some embodiments, some or all of the blocks of the method 200 are performed as part of a medical procedure for treating the patient for a fluid overload condition. The method 200 can be used as a primary, standalone therapy for treating fluid overload, or can be used in combination with other therapies (e.g., as a post-primary therapy to reduce the likelihood of re-hospitalization). The method 200 can be performed in any suitable setting, such as an inpatient setting or an outpatient setting. In embodiments where the method 200 is performed as an outpatient therapy, the overall duration of the method 200 can be reduced (e.g., to no more than 10 hours, 5 hours, 4 hours, 3 hours, 2 hours, or 1 hour).

The method 200 illustrated in FIG. 2 can be modified in many different ways. For example, any of the blocks of the method 200 can be omitted, such as blocks 204 or 206. In some embodiments, block 204 is omitted so that the method 200 controls hydration fluid infusion but not diuretic delivery, or so that the method 200 does not involve any diuretic delivery at all. Similarly, block 206 can be omitted so that the method 200 controls diuretic delivery but not hydration fluid infusion, or so that the method 200 does not involve any hydration fluid infusion at all. As another example, some or all of the blocks 200 of the method 200 can be performed in a different order and/or repeated (e.g., any of blocks 202, 204, 206, and/or 208). In a further example, the method 200 can optionally include additional blocks not shown in FIG. 2 (e.g., causing delivery of additional medications, obtaining parameters other than urine output rate, etc.).

The present technology can provide many advantages for treating fluid overload and/or managing patient fluid levels. For example, embodiments of the present technology have been shown to consistently reduce the fluid volume in patients faster and safer than conventional treatment systems and methods. For example, whereas conventional methods can typically take at least five days to remove 4-5 L of net fluid volume, embodiments of the present technology have been shown to remove 4-5 L liters of net fluid volume in no more than 24 hours. Additionally, embodiments of the present technology have also been shown to remove significant amounts of salt via high sodium urine from patients. This can reduce the likelihood of the patient reaccumulating fluid after discharge, which can lead to reductions in rehospitalization rates. Moreover, embodiments of the present technology can automatically and continuously monitor urine output, hydration fluid infusion, and/or diuretic delivery to mitigate patient safety concerns (e.g., over-diuresis and/or hypotension) during the treatment procedure.

Embodiments of the present technology can provide various benefits, such as any of the following: (i) optimizing net fluid volume removal and/or net sodium removal; (ii) reducing the time needed to achieve desired net fluid removal by allowing physicians to use higher diuretic dosages and/or dosage rates earlier in treatment compared to conventional treatments; (iii) avoiding or reducing risk of adverse events such as over-diuresis, dehydration, acute kidney injury, and/or intravascular depletion; (iv) quickly assessing if a patient is diuretic resistant; and (v) providing a record of treatment data. Embodiments of the present technology may obtain an average net fluid removal rate (e.g., average urine output rate minus average hydration fluid infusion rate) of at least 225 mL/hr, which provides 3.4 L per day of net fluid volume removal based on introducing 2 L of fluid per day orally or through IV infusion. This rate of fluid removal, while replacing sodium, may reduce the overall length of stay and/or provide enhanced decongestion.

Various aspects of one or more embodiments of the present technology can be based at least partially on one or more models (“model(s)”), such as artificial intelligence (AI) and/or machine learning (ML) models. Individual ones of these models can be trained using historical treatment data from one or more other patients and configured to determine and/or predict information about the patient receiving treatment, and/or otherwise inform the system's and/or the user's decisions regarding the patient's therapy. For example, in some embodiments the model(s) are configured to determine and/or predict information associated with diuretic delivery during fluid therapy. Individual ones of the model(s) can be configured to (i) predict the diuretic dose to elicit the desired urine output response from the patient, (ii) predict the occurrence of therapy re-ramps and/or automatically re-ramp the patient's therapy, and/or (iii) identify and/or select the diuretic(s) most likely to elicit the desired fluid removal response. Additionally, or alternatively, the model(s) are configured to determine and/or predict information associated with hydration fluid delivery during fluid therapy. For example, the model(s) can be configured to (i) predict the risk of the patient tolerating/not tolerating a given hydration fluid delivery rate, (ii) predict the likelihood of the patient's urine output exceeding a threshold value (e.g., falling below a threshold value), and/or (iii) adjust the patient hydration fluid delivery rate to improve the patient's urine output. In these and other embodiments, the model(s) are configured to determine and/or predict information associated with altering the patient's fluid therapy, including one or more steps, blocks, and/or protocols associated therewith. For example, the model(s) can be configured to (i) determine information associated with a decision to stop the patient's fluid therapy to guide a user's decision regarding the same, (ii) predict a readmission risk for the patient once the patient's fluid therapy has ended, (iii) recommend and/or determine oral diuretic dosage information associated with the patient, and/or (iv) identify patients not expected to respond to fluid therapy. In further embodiments, one or more of the model(s) are configured to determine, before beginning and/or during fluid therapy, a likelihood of the patient experiencing one or more adverse events during the fluid therapy. In some embodiments, the model(s) are configured to determine a time until a hydration fluid source and/or a diuretic source is expected to be empty and/or a time until a urine collection container is expected to be full.

Generally, the model(s) are expected to improve the effectiveness of the fluid therapy steps/blocks/protocols described herein. In some embodiments, the model(s) are expected to improve a specific patient's response to the fluid therapy steps/blocks/protocols described herein. In further embodiments, the model(s) are expected to optimize (e.g., maximize) a patient's response to the fluid therapy steps/blocks/protocols described herein in real-time, for example, based on data received from the patient associated with the patient's response to the fluid therapy.

### II. Obtaining Urine Characteristics, and Associated Systems, Devices and Methods

FIG. 3 is a partially schematic illustration of a urine flow cartridge 301 configured in accordance with embodiments of the present technology. The urine flow cartridge 301 can also be referred to as “cartridge 301,” “urine cartridge 301,” “flow cartridge 301,” “sensing cartridge 301,” “integrated cartridge 301,” “flow cell 301,” and the like. At least some aspects of the cartridge 301 can be generally similar or identical in structure and/or function to the cartridge 101 of FIG. 1.

The cartridge 301 can define a flow channel 302. The flow channel 302 can include a fluid inlet 304 and a fluid outlet 306. The fluid inlet 304 can be fluidly coupled to the patient via, e.g., the fluid line 119, or otherwise configured to receive fluid (e.g., urine) from the patient. The fluid outlet 306 can be fluidly coupled to the collection container 112 and/or another suitable fluid outflow location (e.g., waste fluid collection). Although the cartridge 301 includes a single flow channel 302 having one inlet 304 and one outlet 306 in the embodiment illustrated in FIG. 3, in other embodiments the cartridge 302 can include more flow channels 301 (at least, e.g., two, three, four, etc.) and/or individual ones of the flow channels 302 can have respective inlets 304 and/or outlets 306 or share an inlet 304 and/or outlet 306 with one or more other flow channels. In operation, fluid (e.g., urine) that enters the cartridge 301 via the fluid inlet 304 can flow through all or a portion of the flow channel 302, toward and/or out through the fluid outlet 306 and/or into the container 112.

The cartridge 301 can further include one or more sensors 314 (individually identified as a first sensor 314a, a second sensor 314b, a third sensor 314c, a fourth sensor 314d, and an n-th sensor 314n) positioned at various locations along the flow channel 302. Individual ones of the sensors 314 can be at least generally similar or identical in structure and/or function to the sensors 114 of FIG. 1. Each of the sensors 314 can include one or more respective sensing elements 316 (individually identified as a first sensing element 316a of the first sensor 314a, a second sensing element 316b of the second sensor 314b, a third sensing element 316c of the third sensor 314c, a fourth sensing element 316d of the fourth sensor 314d, and an n-th sensing element 316n of the n-th sensor 314n) configured to detect one or more characteristics (including, e.g., output/flow rate, temperature, conductivity, concentration, partial pressure of oxygen, etc.) of the fluid (e.g., urine) flowing through the flow channel 302. Individual ones of the sensing elements 316 can be positioned at least partially within the flow channel 302, positioned around at least a portion of an exterior of the flow channel 302, contactless, and/or otherwise positioned to detect one or more characteristics of the fluid flowing through the flow channel 302. In at least some embodiments, for example, the sensors 314 include one or more conductivity sensors, temperature sensors, oxygen sensors, flow sensors, etc. In these and other embodiments, the sensing elements 316 include one or more electrically conductive contacts, one or more thermistors, one or more light sources (e.g., LEDs, infrared light sources), etc. Each of the sensing elements 316 and sensors 314 can be communicatively coupled to the controller 140 so that readings from the sensing elements 316 and sensors 314 can be communicated to the controller 140 to, e.g., analyze, monitor, and/or otherwise obtain characteristics of the fluid. The obtained characteristics can be used to start, stop, or adjust the patient's fluid therapy, as described herein.

In operation, urine flows from the fluid line 119 and contacts one or more of the sensing elements 316 as it progresses through the flow channel 302 from the inlet 304 toward the outlet 306 and to the container 112. In some embodiments, the first sensor 314a is a conductivity sensor, the second sensor 314b is a temperature sensor, and the third sensor 314c is an oxygen sensor. In such embodiments, the first sensor 314a can include two electrodes spaced apart from one another. The sensors 314 are operably coupled to the controller 140, and thus the signals from the sensors 316 are communicated to the controller 140 and used to adjust fluid therapy for the patient. The controller 140 can process the signals received, e.g., to obtain a first derivative (e.g., rate of change), second derivative (e.g., change in the rate of change), etc. As described herein, adjustments to fluid therapy can include adjusting (e.g., increasing or decreasing) one or both of diuretic dosage rate and hydration fluid infusion rate.

FIG. 4 is a partially schematic illustration of a cartridge 401 configured in accordance with embodiments of the present technology. At least some aspects of the cartridge 401 can be at least generally similar or identical in structure and/or function to the cartridge 101 of FIG. 1 and/or the cartridge 301 of FIG. 3. For example, the cartridge 401 defines a fluid channel 402 having a fluid inlet 404 fluidly coupled to the patient via, e.g., the fluid line 119, and a fluid outlet 406 fluidly coupled to the container 112 and/or another fluid outflow location. The cartridge 401 further includes fluid (e.g., urine) conductivity sensors 414 and a fluid (e.g., urine) temperature sensor 418. The conductivity sensor 414 can include a pair of electrically conductive contacts 416a-b spaced apart from one another along the fluid channel 402 to obtain a conductivity of the fluid flowing between the pair of electrically conductive contacts 416a-b. The pair of electrically conductive contacts 416a-b can be contactless or positioned within the fluid channel 402. In contactless embodiments, one of the pair of electrically conductive contacts 416a, 416b can generate an electrical field that induces a current in the other of the pair of electrically conductive contacts 416a, 416b. Changes to the type, composition, etc. of the fluid within the fluid channel 402 can change one or more aspects (e.g., amplitude, voltage, etc.) of the induced current and this change in the induced current can be used to determine one or more characteristics (e.g., conductivity, resistance, etc.) of the fluid within the fluid channel 402. The temperature sensor 418 can be positioned between (e.g., equidistant from) the pair of electrically conductive contacts 416a-b. Conductivity varies with temperature, so positioning the temperature sensor 418 between the pair of electrically conductive contacts 416a-b allows the temperature sensor 418 to detect a temperature that is generally or substantially an average of the temperature of the fluid at or near each of the pair of electrically conductive contacts 416a-b. In some embodiments, the temperature sensor 418 is positioned upstream or downstream from the pair of electrically conductive contacts 416a-b. However, positioning the temperature sensor 418 between the pair of electrically conductive contacts 416a-b allows the pair of electrically conductive contacts 416a-b to be spaced apart to reduce the variability in the measurements by these contacts 416a-b while also reducing (or even minimizing) the size of the cartridge 401 compared to, e.g., embodiments in which the temperature sensor 418 is positioned upstream or downstream from the pair of electrically conductive contacts 416a-b. The controller 140 can adjust the conductivity data based at least partially on temperature data from the temperature sensor 418. In at least some embodiments, the controller 140 can determined an estimated and/or indicated sodium content of the patient's urine based at least in part on temperature data from the temperature sensor 418 and conductivity data from the conductivity sensor 414.

The fluid line 119, or a portion thereof, can have an outer diameter (OD) larger than its inner diameter (ID). For example, the fluid line 119 can have an OD of at least 0.25 inches and/or an ID of at least 0.125 inches. In some embodiments, the fluid line 119 is thinner than other (e.g., conventional) fluid lines to provide a more accurate reading of, e.g., the urine characteristics described herein.

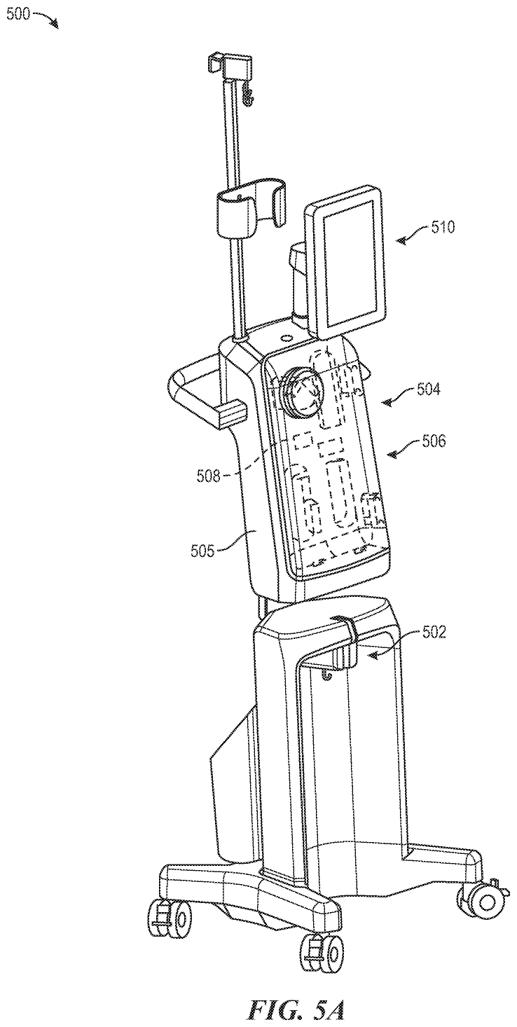

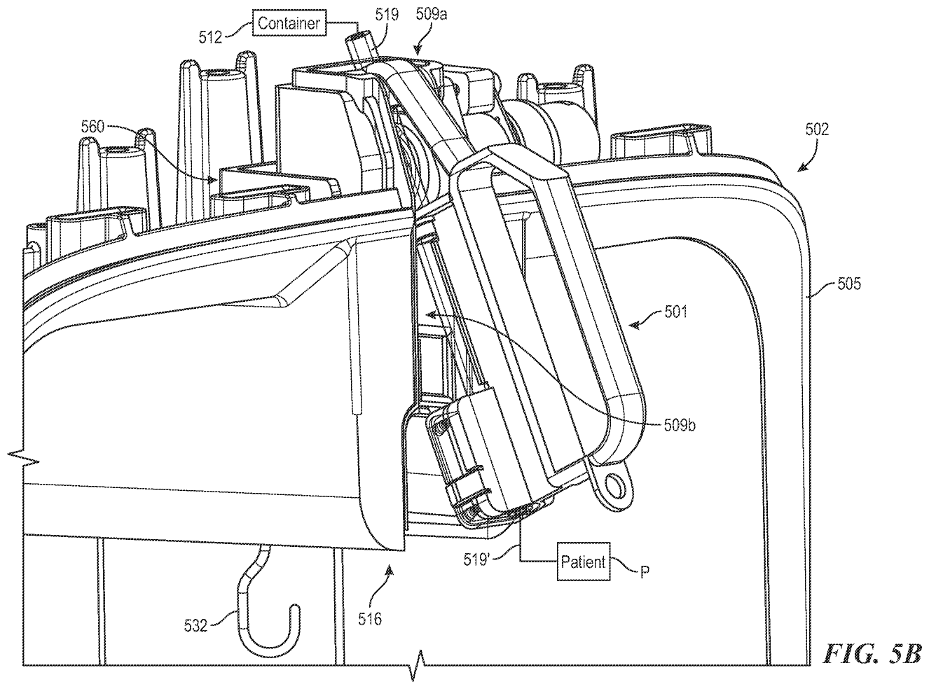

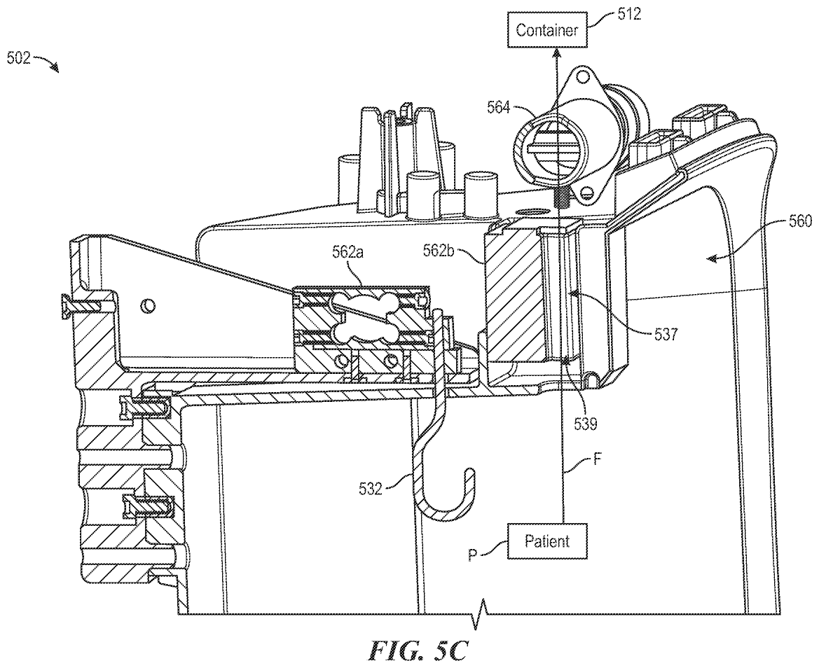

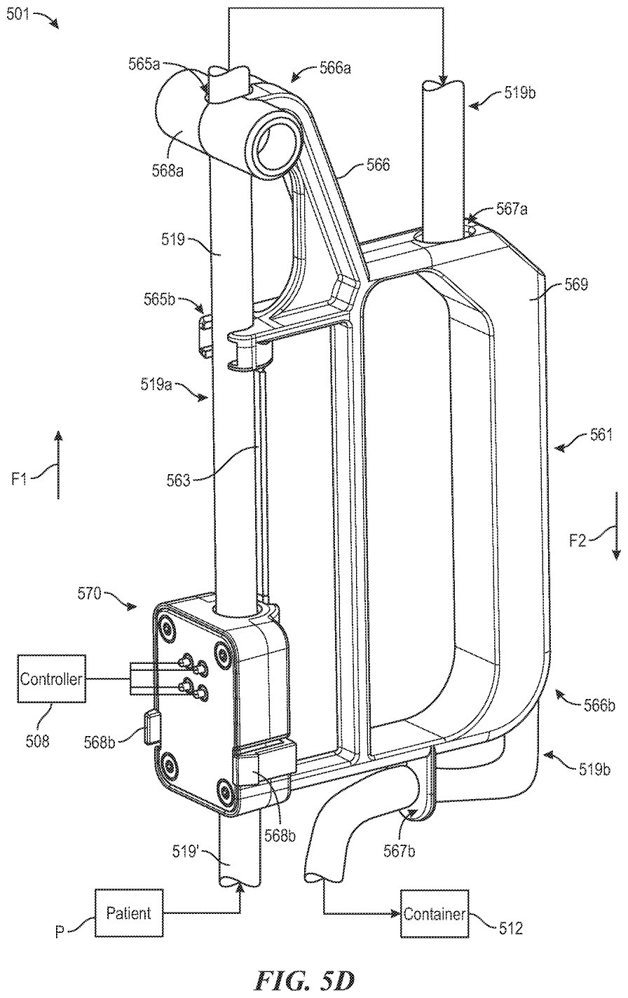

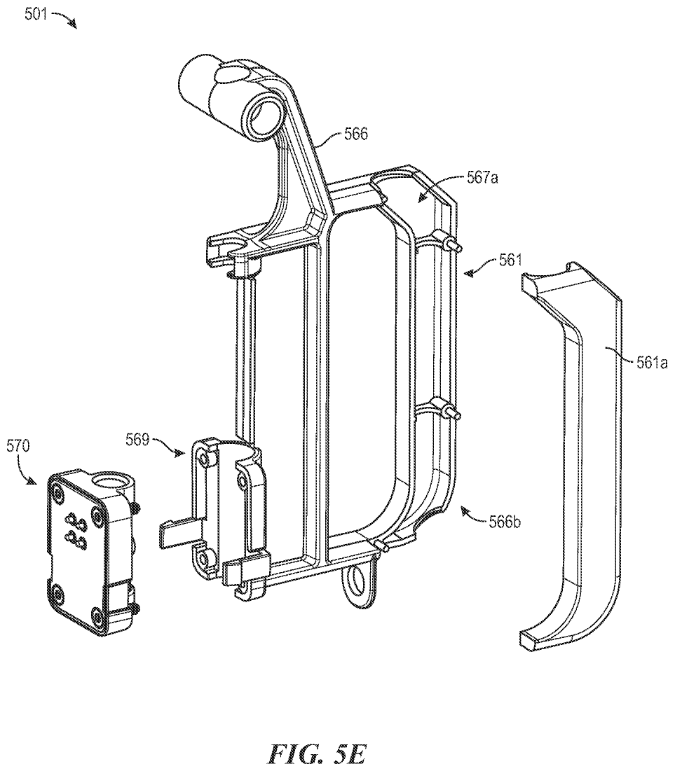

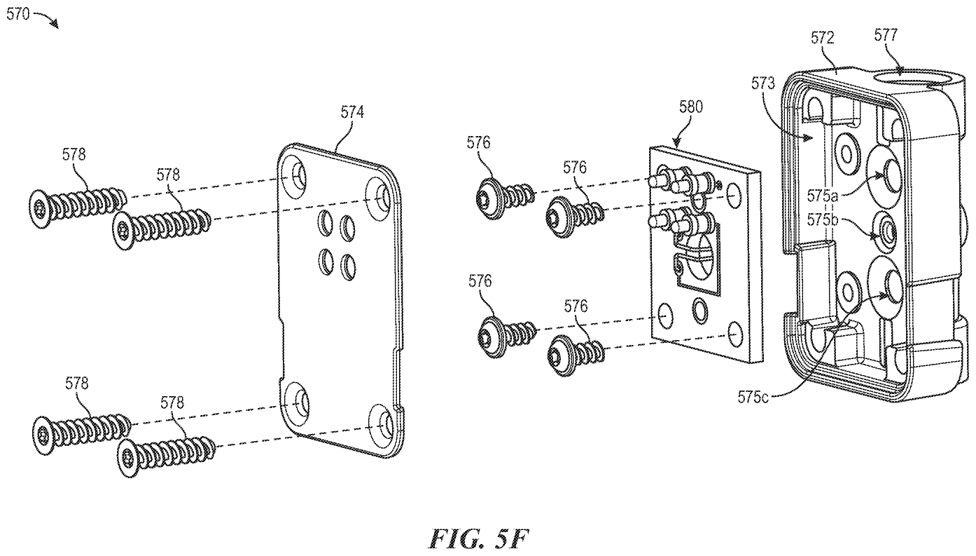

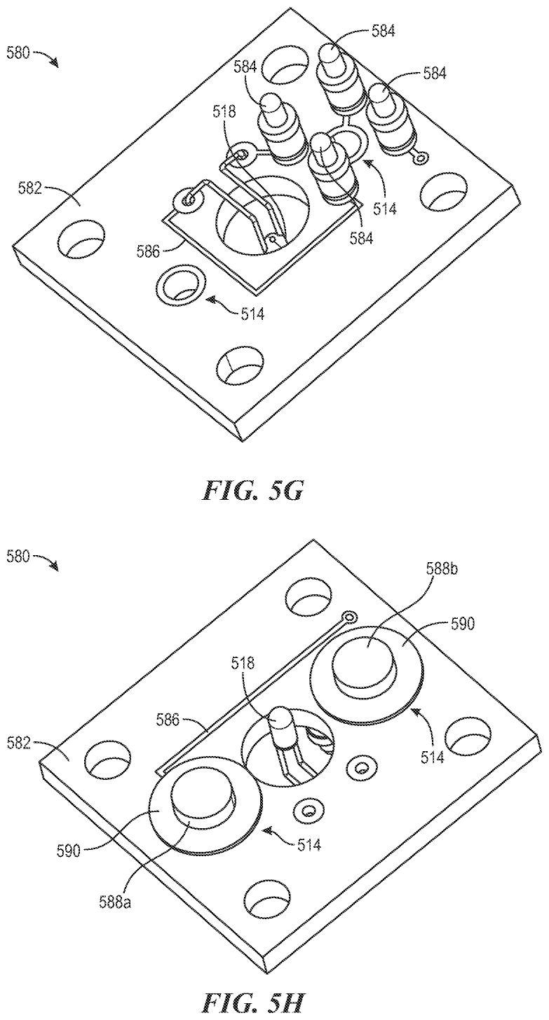

FIG. 5A is a perspective view of another urine collection system 500 (“system 500”) configured in accordance with embodiments of the present technology. The system 500 can include at least some aspects that are generally similar or identical in structure and/or function to one or more of the embodiments described herein (e.g., the systems 100 of FIG. 1), such as one or more of the components for monitoring and/or managing fluid levels previously described with reference to FIG. 1. Additionally or alternatively, any of the features of the embodiments of FIGS. 5A and 5B can be combined with each other and/or with any of other systems and devices described herein (e.g., the system 100 of FIG. 1).

Referring to FIG. 5A, the system 500 includes a urine collection and monitoring system 502 (“urine system 502”), an automated hydration fluid infusion system 504 (“hydration system 504”), an automated diuretic infusion system 506 (“diuretic system 506”), a controller or control system 508 (“controller 508”), and a display or input/output unit 510 (“display 510”). The controller 508 can be operably coupled to each of the urine system 502, hydration system 504, diuretic system 506, and/or display 510. The system 500 can further include a console or structure 505 (“console 505”) that incorporates, houses, and/or otherwise supports all or portions of the urine system 502, hydration system 504, diuretic system 506, the controller 508, and/or the display 510. Similar to the embodiments described above, the urine system 502 collects and monitors urine from a patient while the automated hydration fluid infusion system 504 automatically delivers fluid to the patient and/or the automated diuretic infusion system 506 automatically delivers a diuretic to the patient based on, in part, data obtained from the urine system 502. For example, as described herein, the amount of diuretic and/or hydration fluid provided to the patient is based on urine output from the patient, the patient's urine conductivity, and/or the patient's urine oxygen content, as measured via the urine system 502.

FIG. 5B is a partially-schematic perspective view of the urine system 502. The urine system 502 can include a urine cartridge 501 and a urine flow assembly 560. The urine cartridge 501 and the urine flow assembly 560 can together be referred to herein as a flow control assembly 516. The urine flow assembly 560 can include a container mounting component 532 (“mounting component 532”). In the illustrated embodiment, the mounting component 532 includes a coupler (e.g., a hook) from which a container 512 (e.g., a urine bag or the container 112, FIG. 1) can hang or be supported. The mounting component 532 is movable between a first unstressed position and a second stressed position when the mounting component 532 is supporting a weight of the container 512. As such, when in the second position or not in the first position, the mounting component 532 can indicate the presence of the container 512 thereon. When in the second position, the mounting component 532 can engage components of the urine flow assembly 560, e.g., by activating one or more sensors to monitor and/or determine a urine output rate of the patient.

The urine cartridge 501 can be detachably coupled to the urine flow assembly 560. For example, the urine flow assembly 560 can include one or more receiving features 509 (identified by reference numbers 509a and 509b) configured to receive the urine cartridge 501. In the illustrated embodiment, the urine flow assembly 560 includes a pivotal receiving feature 509a and a slot receiving feature 509b, which together can couple the urine cartridge 501 to the urine flow assembly 560. The pivotal receiving feature 509a can pivotally engage the urine cartridge 501 such that the urine cartridge 501 can pivot toward and/or at least partially into the slot receiving feature 509b to engage (e.g., operably engage) the urine flow assembly 560. The urine cartridge 501 can be coupled to a proximal fluid line 519′ configured to receive urine and/or other fluid from the patient P and a distal fluid line 519 configured to direct urine to the container 512. When coupled to the urine flow assembly 560, the urine cartridge 501 can position and orient the distal fluid line 519 relative to aspects of the urine flow assembly 560 to enable urine flow measurement and to provide a urine output (e.g., an average urine output rate). In some embodiments, the distal fluid line 519 is coupled (e.g., adhered) to the urine cartridge 501 prior to attached the urine cartridge 501 to the console 505 at the receiving feature 509. Additional details regarding the engagement between the urine cartridge 501 and the receiving features 509 are described in U.S. Pat. No. 11,633,137, filed Jun. 8, 2022, the entirety of which is hereby incorporated by reference herein. In some embodiments, the proximal fluid line 519′ can be coupled to the container 512 (rather than the patient P, as shown in FIG. 5B) and the distal fluid line 519 can be coupled to the patient P (rather than the container 512, as shown in FIG. 5B). In such embodiments, the urine cartridge 501 can operate as described herein, e.g., to direct urine flow from the patient P to the container 512 but the flow through at least the portion of the distal fluid line 519 that engages the urine flow assembly 560 can be in an opposite (e.g., downward) direction.

FIG. 5C is a partially schematic, perspective cross-sectional view of the system 502. Fluid F (e.g., urine) from the patient P can flow through a portion of the urine flow assembly 560 and into the container 512 via the distal fluid line 519 (FIG. 5B). The urine flow assembly 560 can include one or more fluid sensors 562 operable to measure and/or determine the flow of the fluid F through the urine system 502. In the illustrated embodiment, the urine flow assembly 560 includes a first fluid sensor 562a and a second fluid sensor 562b. The first sensor 562a can include a load cell and be configured to measure or generate (e.g., on a continuous basis) first sensor data including a weight of the container 512 when coupled to the mounting component 532. The first sensor data (e.g., the weight and/or the change in weight of the container 512) can be used to generate a first patient urine output (e.g., an average volumetric flow rate). The second sensor 562b can include a flow sensor and be configured to measure or generate (e.g., on a continuous basis) second sensor data including a flow of the fluid F through the fluid line. The second sensor data can be used to generate a second patient urine output (e.g., an average volumetric flow rate).

The second sensor 562b can include a groove 537 (e.g., a U-shaped groove) that at least partially defines a slot or channel 539 (“slot 539”) that receives a portion of the distal fluid line 519. The slot 539 is further defined on an opposing side by a portion of the urine cartridge 501 when coupled to the urine flow assembly 560. Referring additionally to FIG. 5B, when the urine cartridge 501 is coupled to the system 502 and/or console 505 (FIG. 5B), the urine cartridge 501 and flow sensor 562b can position and/or orient the fluid line 519 within the slot 539 to ensure an accurate and reliable flow measurement. In the illustrated embodiment, for example, the urine cartridge 501 can be configured to press and/or hold the portion of the distal fluid line 519 against the flow sensor 562b, which can improve the accuracy of the urine output measured via the flow sensor 562b. Stated differently, if the distal fluid line 519 is not properly set within the slot 539, or if length of distal fluid line 519 extending from the urine cartridge 501 to the container 512 is improper (e.g., too short), then the flow measurement via the flow sensor 562b can be less accurate and/or less consistent between measurements. For example, if the distal fluid line 519 extending from the urine cartridge 501 to the container 512 is too short, the container 512 may add additional stress on and/or physically dislodge the urine cartridge which can affect the flow measurement via the flow sensor 562b. In addition, if the distal fluid line 519 extending from the urine cartridge 501 to the container 512 is too short, the container 512 may be pulled by the distal fluid line 519 which can alter the container 512 weight reading of the first sensor 562a leading to inaccurate measurement of fluid flow rate or volume into the container 512.

Referring again to FIG. 5C, the urine system 502 can further include one or more flow control devices 564. The flow control device 564 can include a pinch clamp or valve configured to fully or at least partially regulate fluid flow through the system 502, such as when priming one or more of the fluid lines. The flow control device 564 can also be used to regulate flow if the system 502 determines that the weight of the container 512 is decreasing, e.g., based on the first sensor data from the first sensor 562a, or through user input. In such embodiments, the flow control device 546 may only regulate flow if the second sensor 562b is disabled or non-operational and only the first sensor 562a is operational. During the time the flow control device 564 is closed and there is no flow to the container 512, patient urine output is not measured. However, in such embodiments, the rate and/or volume of urine output can be calculated or estimated based on at least the time of no flow and the resulting flow measurement once flow resumes. In some embodiments, the pinch clamp 564 is configured to shut off flow once the distal fluid line 519 (FIG. 5B) and/or the proximal fluid line 519′ (FIG. 5B) are primed. Priming the fluid lines 519, 519′ can remove any air within these lines and/or otherwise create a continuous (or at least generally continuous) column of fluid therein. This solid column of fluid means that, as soon as the patient excretes urine into the bladder, that volume of fluid is immediately seen as a weight/volume/etc. increase in the container 512. The pinch clamp 564 can shut off flow through these lines to prevent, or at least partially prevent, air from entering the primed fluid line, such as when the fluid lines 519, 519′ are disconnected from the cartridge 501. Additional details regarding priming fluid lines are described in U.S. Pat. No. 11,633,137, filed Jun. 8, 2022, the entirety of which is hereby incorporated by reference herein. The flow control device 564 can regulate the fluid flow without touching the fluid by externally pinching the fluid line. Alternately, the flow control device 546 can be a gate, needle or other type of valve that can regulate fluid flow by being in contact with the fluid. In the illustrated embodiment, the flow control device 564 is positioned upstream from the flow sensor 562b. In other embodiments, the flow control device 564 can be positioned downstream from the flow sensor 562b, and/or have any other suitable positions.

FIG. 5D is a perspective view of the urine cartridge 501. The urine cartridge 501 can include a body 566 having (i) a first or upper end portion 566a, (ii) a second or lower end portion 566b opposite the first end portion 566a, and (iii) a handle 561 at least partially between the first and second end portions 566a,b. The first end portion 566a can include a first urine system coupling feature 568a (“first coupling feature 568a”) and the second end portion 566b can include a second urine system coupling feature 568b (“second coupling feature 568b”). The first coupling feature 568a and the second coupling features 568b can be configured to releasably engage the urine flow assembly 560 to ensure a precise placement of the fluid line 519 relative to the flow sensor 562b (FIG. 5C). In the illustrated embodiment, for example, the first coupling feature 568a can be pivotally received by the pivotal receiving feature 509a (FIG. 5A) and the second coupling feature 568b can be inserted within a correspondingly-shaped recess (not shown) in the console 505 (FIGS. 5A and 5B). The second coupling feature 568b can include one or more tabs having a flared end or other shape configured to matingly engage one or more correspondingly shaped recesses or slots in the console 505.

The urine cartridge 501 can further include a sensor assembly 570, one or more urine line coupling features 565, and/or one or more urine line return features 567. The sensor assembly 570, the one or more urine line coupling features 565, and the one or more urine line return features 567 can receive or otherwise direct fluid F to flow from the patient P to the container 512. Accordingly, the sensor assembly 570, the one or more urine line coupling features 565, and/or the one or more urine line return features 567 can together define a pathway for fluid to flow from the patient P to the container 512.

The sensor assembly 570 can be coupled to the body 566 of the urine cartridge 501 so that, when the urine cartridge 501 is received within the slot receiving feature 509b (FIG. 5B), the sensor assembly 570 can be operably coupled to a controller 540 of the system 500 (e.g., the controller 140 of FIG. 1). The sensor assembly 570 can be configured to receive fluid from the patient P via, e.g., the proximal fluid line 519′. The sensor assembly 570 can be oriented so that fluid from the patient flows vertically and/or upwardly through the sensor assembly 570, in the first direction F1 shown in FIG. 5D, or in another upward-oriented direction within plus or minus 1 degree, 5 degrees, 10 degrees, 15 degrees, 20 degrees, or 30 degrees of the first direction F1. Orienting the sensor assembly 570 in this manner is expected to reduce or even prevent airlocks and/or other gas bubbles from forming within the sensor assembly 570 and/or associated tubing. For example, because gas (e.g., air) is more buoyant than liquid (e.g., urine, water, etc.), directing fluid flow upwardly through the sensor assembly 570 allows gas's naturally buoyancy to urge any gas within the sensor assembly 570 and/or associated tubing to flow upwardly in a same direction as the fluid flow. This creates a tendency for any gas within the sensor assembly 570 and/or associated tubing to flow with or faster than fluid, reducing the likelihood that the gas forms any bubbles or airlocks that reduce or stop fluid flow and/or create false or inaccurate readings from the sensor assembly 570.

The one or more urine line coupling features 565 can be configured to position a first or upstream portion 519a of the fluid line 519 relative to the urine flow assembly 560 (FIG. 5B and 5C) as described previously herein with reference to at least FIGS. 5B and 5C. The upstream portion of the fluid line 519 can direct fluid flow in the first direction F1. In the illustrated embodiment the first urine line coupling feature 565a can include an aperture or opening extending through the first coupling feature 568a and the second urine line coupling feature 565b includes a forked or pinch connector. In other embodiments, one or more of the urine line coupling features 565 can have other suitable configurations that allow the urine line coupling features 565 to position the upstream portion 519a of the fluid line 519 relative to the urine flow assembly 560 (FIG. 5B and 5C). In these and/or other embodiments, the fluid line 519 can be bonded or otherwise adhered (e.g., via adhesive) to the urine cartridge at one or more of the coupling features 565.

The one or more urine line return features 567 can be configured to engage a second or downstream portion 519b of the fluid line 519 to, e.g., facilitate the return of the fluid line 519 to the container 512, including when the container 512 is suspended from the mounting component 532 (e.g., below the urine cartridge 501). The downstream portion 519b of the fluid line 519 can direct fluid flow in a second direction F2 different than (e.g., opposite) the first direction F1, or in another downward-oriented direction within plus or minus 1 degree, 5 degrees, 10 degrees, 15 degrees, 20 degrees, or 30 degrees of the second direction F2. In the illustrated embodiment, the first urine line return feature 567a includes a channel extending through at least a portion of the handle 561, and an opening or ring anchor 567b positioned at the second end portion 566b. The channel 567a can be defined, at least in part, by a removable section 561a of the handle 561. The removable section 561a can be removed from the rest of the body 566 to allow the fluid line 519 to be positioned within the channel 567a.

In some embodiments, the urine cartridge 501 includes a fluid line engagement feature 563 (“engagement feature 563”) that can at least partially define the slot 539 (FIG. 5C) when the urine cartridge 501 is coupled to the console 505. The engagement features 563 can be positioned and/or otherwise configured to operably engage or abut the fluid line 519 with the urine flow assembly 560. As previously described (e.g., with reference to FIG. 5C), the urine cartridge 501 can position and/or orient the fluid line 519 within the slot 539 to ensure an accurate and consistent flow measurement. In the illustrated embodiment, the engagement feature 563 includes a protrusion or tab extending from the body 566 to press or otherwise operably engage the distal fluid line 519 within the slot 539 and/or limit bending, flexing, or an otherwise undesired orientation of the distal fluid line 519 when the urine cartridge 501 is coupled to the console 505. In other embodiments, the engagement feature 563 can include a series of protrusions or tabs, or have another structure configured to operably position the distal fluid line 519 relative to the console 505 when the urine cartridge 501 is coupled to the console 505.

FIG. 5E is a partially-exploded perspective view of the urine cartridge 501. The distal and proximal fluid lines 519, 519′ (FIG. 5D) are omitted for illustrative clarity. The removable section 561a of the handle 561 can be disconnected from the rest of the body 566 to allow a user to access the channel 567a to, for example, position a portion of the distal fluid line 519 (FIG. 5D) within the channel 567a. The body 566 can define a receiving area 569 configured to releasably receive the sensor assembly 570.

FIG. 5F is a partially exploded view of the sensor assembly 570. The sensor assembly 570 can include a sensor assembly housing 572, a cover plate 574, one or more first fasteners 576, one or more second fasteners 578, and a sensor board 580. The sensor assembly housing 572 can define an interior 573, one or more sensing apertures 575 (individually identified as first, second, and third sensing apertures 575a-c, respectively), and a fluid conduit 577. The interior 573 can be configured to receive the sensor board 580. The fluid conduit 577 can be configured to be positioned in-line with the distal fluid 519 to, e.g., receive fluid (e.g., urine) flow from the patient. Accordingly, urine and/or other fluid from the patient can flow through the fluid conduit 577, through the upstream portion 519a and the downstream portion 519b of the distal fluid line 519 (FIG. 5D), and enter the container 512 (FIG. 5D). The one or more sensing apertures 575 can define respective openings into the fluid conduit 577. When the sensor board 580 is positioned within the interior 573, various sensing elements of the sensor board 580 can be positioned at least partially within the fluid conduit 577, via the sensing apertures 575, or otherwise operably positioned relative to the fluid conduit 577 to obtain one or more characteristics associated with the fluid within the fluid conduit 577. In at least some embodiments, for example, one or more sensors carried by the sensor board 580 can be contactless and the one or more sensing apertures 575 can be minimized or omitted.

The one or more first fasteners 576 can be configured to couple the sensor board 580 to the sensor assembly housing 572 within the interior 573. The one or more first fasteners 576 can, accordingly, at least partially prevent the sensor board 580 from moving relative to the one or more sensing apertures 575. The one or more first fasteners 576 can include screws (shown in the embodiment illustrated in FIG. 5F), pins, rivets, adhesives, and/or other suitable fasteners. In some embodiments, the one or more first fasteners 576 can be omitted and the sensor assembly housing 572 and/or the sensor board 580 can be configured to be coupled to one another via an interference fit, correspondingly shaped tabs and tab-receiving features configured to matingly engage with one another, etc.

The cover plate 574 can be coupled to the sensor assembly housing 572, via the one or more second fasteners 578, to cover or seal the interior 573. The one or more second fasteners 578 can include screws (shown in the embodiment illustrated in FIG. 5F), pins, rivets, adhesives, ultrasonic welds, and/or other suitable fasteners or fastening techniques. In some embodiments, the one or more second fasteners 578 can be omitted and the sensor assembly housing 572 and/or the cover plate 574 can be configured to be coupled to one another via an interference fit, correspondingly-shaped tabs and tab-receiving features configured to matingly engage with one another, etc.

FIGS. 5G and 5H are top and bottom perspective views, respectively, of the sensor board 580. The sensor board 580 can include a body 582, a fluid (e.g., urine) conductivity sensor 514, a fluid (e.g., urine) temperature sensor 518, one or more connectors 584, and one or more interconnectors 586. The conductivity sensor 514 can include a pair of electrically conductive contacts 588a-b (FIG. 5H) spaced apart from one to obtain a conductivity of the fluid flowing between the pair of electrically conductive contacts 588a-b. The pair of electrically conductive contacts 588a-b can be contactless, positioned within the fluid conduit 577 (FIG. 5F), and/or otherwise positioned to obtain one or more characteristics from the fluid within the fluid conduit 577, as described previously herein. The pair of electrically conductive contacts 588a-b can include gold, carbon, and/or other suitable electrically conductive contacts. Gold is less likely to develop biofilms than carbon electrodes, but carbon electrodes may be better suited when operating for longer periods of time (e.g., more than 1 day, two days, three days, four days, 1 week, 2 weeks, etc.). One or more sealing elements 590 (e.g., O-rings, adhesives, etc.) can be positioned at least partially around the contacts 588a-b. The sealing elements 590 can seal one or more of the sensing apertures 575 (FIG. 5F). The temperature sensor 518 can be positioned generally adjacent to the pair of electrically conductive contacts 588a-b. For example, the temperature sensor 518 can be positioned upstream, downstream, between, and/or otherwise within 6 inches, 5 inches 4 inches, 3 inches, 2 inches, 1 inch, 0.75 inches, 0.5 inches, 0.25 inches, 0.1 inches, 0.01 inches, or another suitable distance of one or both of the pair of electrically conductive contacts 588a-b. The temperature sensor 518 can include a glass bead thermistor and/or one or more other suitable temperature sensors. In some embodiments, one or more sealing elements can be positioned at least partially around the temperature sensor 518. In other embodiments, adhesive (e.g., UV-cured adhesive) and/or other suitable materials can be used to form a substantially fluid-impermeable seal between the temperature sensor 518 and the sensing aperture 575 associated with the temperature sensor 518. The temperature sensor 518 can have a tight fit in the corresponding sensing aperture 575 that prevents, or at least partially prevents, adhesive and/or other sealing elements from migrating into the fluid conduit 577.

The distance (e.g., center-to-center distance) between conductive contacts 588a-b can affect the accuracy of the conductivity measurement, with larger distances often providing more accurate measurements. In the illustrated embodiment, the distance (e.g., center-to-center distance) between conductive contacts 588a-b is 0.44 inches, which can allow the sensor assembly 570 to provide accurate measurements while maintaining a generally compact form-factor. In other embodiments, the distance between the conductive contacts 588a-b can be greater than 0.44 inches, such as up to 0.88 inches, 1 inch, 2 inches, etc. These greater distances may improve the accuracy of the sensor assembly's measurements and/or reduce the variability in the signal from sensor to sensor, but are also expected to increase the size of the sensor assembly 570.

Conductivity measures can also vary with temperature, so positioning the temperature sensor 518 between the pair of electrically conductive contacts 516a-b allows the temperature sensor 518 to detect a temperature that is generally or substantially an average of the temperature of the fluid at or near each of the pair of electrically conductive contacts 516a-b. Positioning the temperature sensor 518 between the pair of electrically conductive contacts 516a-b can also allow the pair of electrically conductive contacts 516a-b to be spaced apart to reduce the variability in the measurements by these contacts 516a-b while also reducing (or even minimizing) the size of the cartridge 501 compared to, e.g., embodiments in which the temperature sensor 518 is positioned upstream or downstream from the pair of electrically conductive contacts 516a-b. The controller 540 (FIG. 5D) can adjust the conductivity data based at least partially on temperature data from the temperature sensor 518. In at least some embodiments, the controller 540 can determined an estimated and/or indicated sodium content of the patient's urine based at least in part on temperature data from the temperature sensor 518 and conductivity data from the conductivity sensor 514. The connectors 584 can include contact pins or other structures configured to communicatively couple the conductivity sensor 514 and/or the temperature sensor 518 to the controller 540, e.g., as shown in FIG. 5D. The one or more interconnectors 586 can include wires, traces, etc. configured to operably couple the conductivity sensor 514 and/or the temperature sensor 518 to the connectors 584.

FIG. 5I is a perspective cross-sectional view of the urine cartridge 501. When the sensor assembly 570 is coupled to the urine cartridge, the conductivity sensor 514 and the temperature sensor 518 can be positioned at least partially within the fluid conduit 577 to, e.g., obtain one or more characteristics of fluid (e.g., urine) received from the patient. The fluid conduit 577 can have a first end 577a and a second end 577b opposite the first end 577a. As shown in FIG. 5I, the second end 577b can be positioned above the first end 577a. the first end 577a can be coupled to the proximal fluid line 519′ to receive fluid from the patient P. The second end 577b can be coupled to the distal fluid line 519 to direct fluid toward the collection container 512 (FIG. 5B). That is, fluid can (i) enter the fluid conduit 577 at the first end thereof 577a via the proximal fluid line 519′ and (ii) flow upwardly in the first direction F1 out of the fluid conduit 577 and into the distal fluid line 519 at the second end 577b of the fluid conduit 577. The fluid conduit 577 can have a first inner diameter D1 greater than a second inner diameter D2 of the distal fluid line 519 and/or the proximal fluid line 519′. In some embodiments, the increased inner diameter D1 allows the electrical field lines (not shown) to form (e.g., fully form) between the conductive contacts of the conductivity sensor 514 without interference from the walls of the fluid conduit 577. The sealing elements 590 can prevent, or at least partially prevent, fluid within the conduit 577 from leaking into the sensor assembly 570 (via, e.g., the sensing apertures 575).

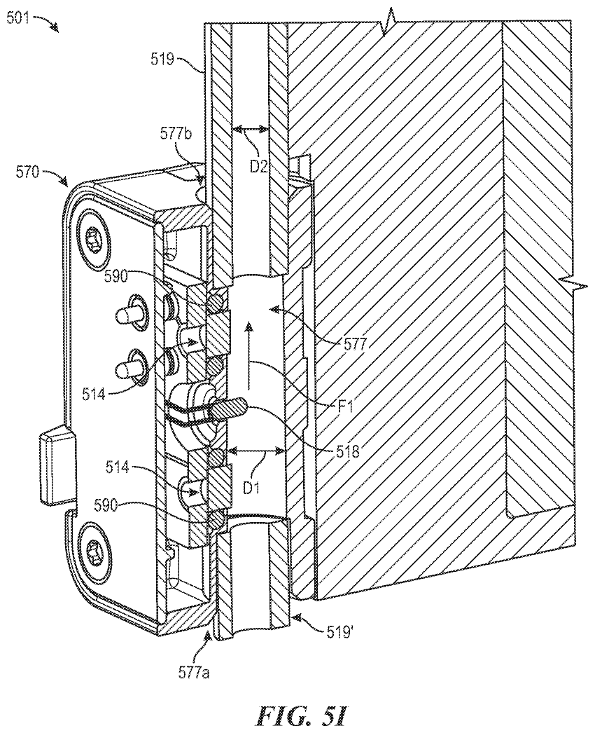

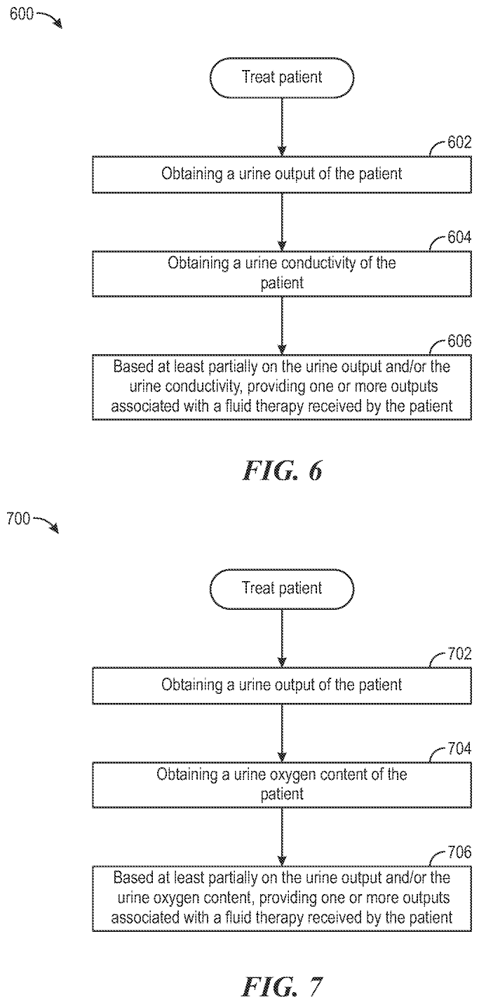

FIG. 6 is a block diagram illustrated a method 600 of providing outputs associated with a patient's fluid therapy based at least partially on a urine output (e.g., a urine output rate) and a urine conductivity of the patient, in accordance with embodiments of the present technology. The method 600 is illustrated as a series of steps, acts, processes, process portions, and/or blocks 602-606. At least some of the blocks 602-606 can be performed by a fluid management system and/or one or more components thereof, such as the system 100 and/or the controller 140 of FIG. 1, and/or the system 500 and/or the controller 508 of FIG. 5A.

At block 602, the method 600 includes obtaining (e.g., detecting or determining) a urine output of the patient. In some embodiments, obtaining the urine output of the patient includes measuring a urine output rate of the patient via, e.g., one or more flow rate sensors. The flow rate sensors can be included in a cartridge, such as one or more of the cartridges 101, 301, 401, 501 described previously herein. Additionally or alternatively, the urine output rate can be obtained via one or more sensors configured to detect an amount (e.g., a weight, volume, etc.) of urine in the collection container 112 (FIG. 1).

At block 604, the method 600 includes obtaining a urine conductivity of the patient. Urine conductivity is highly correlated with urine sodium levels and is thus a good surrogate for urine sodium detection. Additionally, measuring conductivity is less expensive and generally easier relative to measuring sodium concentration directly and provides a signal that is, e.g., more robust and/or less noisy. The sodium concentration of excreted urine is an important measure of health of a heart failure patient undergoing fluid therapy. For example, one of the functions of the kidney is to vary the sodium content of excreted urine in order to maintain serum sodium at a consistent level. One of the drivers of worsening heart failure is the derangement of the patient's native sodium regulation mechanisms. When urine sodium concentration drops below a certain level (e.g., 75 millimoles per liter (“mmol/L”)), this may indicate that the kidneys are retaining sodium to maintain serum sodium levels. If urine sodium concentration rises above a certain level (e.g., 100 mmol/L), this may indicate that the kidneys are excreting excess sodium to maintain appropriate serum sodium levels.

During fluid therapy, sodium containing fluid (e.g., hydration fluid, such as 0.9% saline) can be infused to the patient based at least partially on the patient's urine output. If it is determined (e.g., via the controller 140; FIG. 1) that the patient's urine sodium concentration is dropping, and normal hydration fluid amounts are infused, there is a potential danger that the patient could be pushed into a serum sodium positive state. Thus, if urine sodium drops, the fluid therapy system (e.g., the controller 140) may suggest modifications to the therapy, and/or automatically adjust the therapy, to maintain safety and/or prevent harm to the patient. If, on the other hand, the patient's urine sodium concentration is high, the fluid therapy system may not make modifications, as the patient's kidney is tolerating therapy well. In some embodiments, the hydration fluid infusion rate may be reduced in order to maximize the net fluid and/or net sodium removed from the patient.

In some embodiments, obtaining the urine conductivity includes measuring the urine conductivity using one or more conductivity sensors (e.g., the sensors 414; FIG. 4). The urine conductivity can be measured continuously or intermittently via the one or more conductivity sensors. The conductivity sensors can be included in a cartridge, such as one or more of the cartridges 101, 301, 401, 501 described previously herein. In at least some embodiments, one or more of the conductivity sensors and one or more of the flow rate sensors (block 602) are part of the same cartridge. The measured urine conductivity can be used to provide a corresponding urine sodium concentration. For example, the controller 140 (FIG. 1) can estimate or determine the patient's urine sodium concentration based at least in part on the urine conductivity measurements.

In some embodiments, obtaining the urine conductivity further includes determining a temperature of the patient's urine, e.g., as the urine flows through the conductivity sensor. As described herein (e.g., with reference to FIG. 4), the conductivity of urine varies with temperature, so obtaining the urine temperature can allow for temperature-based compensation of the conductivity signal, which is expected to yield more accurate urine conductivity readings.

In some embodiments, prior to obtaining the urine conductivity, the method 600 includes calibrating the one or more urine conductivity sensors and/or one or more urine sodium concentration sensors. For example, in some embodiments fluid having a known electrolyte content (e.g., saline solution with a known sodium content) is used to prime and/or flush the fluid line 119 and/or any sensors fluidly coupled thereto. Because the fluid has a known electrolyte content, readings from the urine sodium concentration sensors and/or the urine conductivity sensors can be calibrated during the flushing stage.

In block 606, the method 600 includes providing one or more outputs associated with a fluid therapy received by the patient. Generally, the one or more outputs can include one or more alerts or notifications provided to, e.g., a user or practitioner, and/or one or more modifications or adjustments to the patient's fluid therapy. One or more of the outputs can be based at least partially on the urine output (block 602) and/or a change thereto, and/or the urine conductivity and/or sodium concentration (block 604) and/or a change thereto. For example, if urine sodium concentration (or an indication thereof) drops, as measured indirectly by urine conductivity, the fluid therapy system may suggest one or more modifications to maintain safety and/or prevent harm to the patient. Although described below as providing suggestions, those of ordinary skill in the art will appreciate that, in at least some embodiments, the one or more outputs can cause the system to automatically modify the therapy as described in the suggestion to adjust or optimize the therapy, such as by modifying the level of hydration fluid matching of urine output, modifying the diuretic dosage rate, etc.

If the urine conductivity corresponds to a urine sodium concentration less than or equal to a low urine sodium concentration threshold (e.g., less than or equal to 75 mmol/L, and/or that decreases by at least 5%, 10%, 15%, 20%, 30%, etc. over a predetermined time period of, e.g., at least 15 minutes, 30 minutes, 1 hour, 2 hours, 6 hours, 12 hours, 24 hours, etc.) and the urine output is less than or equal to a low urine output threshold (e.g., a urine output rate of less than 325 mL/hr averaged over the previous 3 hours, an integral debt function where the debt is more than 150 mL over the previous 3 hours, and/or decreases by at least 5%, 10%, 15%, 20%, 30%, etc. over a predetermined time period of, e.g., at least 15 minutes, 30 minutes, 1 hour, 2 hours, 6 hours, 12 hours, 24 hours, etc.), the one or more outputs can include an alert that the patient's urine sodium concentration is low and/or dropping, a prompt to actively monitor urine sodium, serum sodium, and/or other patient electrolytes, and/or a prompt to modify or stop the therapy. Additionally or alternatively, the alert can include one or more suggested therapy modifications, such as a change (e.g., increase) to the hydration fluid infusion rate, the rate or percentage matching of infused saline to urine output, and/or a change (e.g., increase) to the rate of diuretic infusion.

In one example, during block 606, the system can adjust the hydration fluid matching percentage such that the amount of infused sodium in the infused hydration fluid matches the amount of indicated sodium that would be infused in the standard algorithm for the same volume of sodium output, assuming a high urine sodium level (e.g., 135 mmol/L). For instance, using 135 mmol/L of urine sodium as the value to normalize to, if the patient outputs 1,050 mL of urine with a measured, estimated or calculated urine sodium concentration of 67.5 mmol/L, the urine volume with the equivalent sodium content of 135 mmol/L urine is 525 mL (i.e., 1050*67.5/135=525). In this embodiment, the saline volume replaced for 525 mL of “normalized” urine is 250 mL of normal saline, thus the 1,050 mL of urine with a sodium concentration of 67.5 mmol/L would also be replaced with 250 mL of normal saline in order to achieve the equivalent sodium balance. While urine sodium levels that exceed 135 mmol/L could be replaced with higher levels of sodium containing fluid, in other embodiment such replacement could be omitted or performed at a lesser rate to, e.g., maximize net sodium removal.

If the indicated urine sodium concentration is less than or equal to the low urine sodium concentration threshold and the urine output is equal to or greater than a high urine output threshold (e.g., more than 625 mL/hr averaged over the previous 3 hours, and/or increases by at least 5%, 10%, 15%, 20%, 30%, etc. over a predetermined time period of, e.g., at least 15 minutes, 30 minutes, 1 hour, 2 hours, 6 hours, 12 hours, 24 hours, etc.), the one or more outputs can include an alert that the patient's urine sodium concentration is high and/or increasing, a prompt to actively monitor urine sodium, serum sodium, and/or other patient electrolytes, and/or a prompt to modify or stop the therapy. For example, the prompt to modify the therapy can include a prompt to reduce saline matching, reduce the hydration fluid infusion rate, and/or increase the diuretic dosage rate.

If the indicated urine sodium concentration is less than or equal to the low urine sodium concentration threshold and the urine output is between the low urine output threshold (of, e.g., 325 mL/hr) and the high urine output threshold (of, e.g., 625 mL/hr), the one or more outputs can include an alert that the patient's urine sodium concentration is acceptable, a prompt to actively monitor urine sodium, serum sodium, and/or other patient electrolytes, and/or a prompt to continue therapy and/or that therapy modifications are not needed at this time.

If, on the other hand, the indicated urine sodium concentration is high, it suggests that the patient's kidney is tolerating therapy well. More specifically, if the indicated urine sodium concentration is equal to or greater than a high urine sodium concentration threshold (e.g., greater than or equal to 100 mmol/L, and/or increases by at least 5%, 10%, 15%, 20%, 30%, etc. over a predetermined time period of, e.g., at least 15 minutes, 30 minutes, 1 hour, 2 hours, 6 hours, 12 hours, 24 hours, etc.), and the urine output is less than or equal to the low urine output threshold (of, e.g., 325 mL/hr), the one or more outputs can include a prompt to increase the diuretic dosage rate, administer an additional diuretic, and/or stop the patient's therapy.

If the indicated urine sodium concentration is equal to or greater than the high urine sodium concentration threshold (of, e.g., 110 mmol/L) and the urine output is equal to or greater than the high urine output threshold (of, e.g., 625 mL/hr), the one or more outputs can include a prompt to reduce hydration fluid matching to, e.g., maximize fluid and/or salt removal from the patient.

If the indicated urine sodium concentration is equal to or greater than the high urine sodium concentration threshold (of, e.g., 100 mmol/L) and the urine output is between the low urine output threshold (of, e.g., 325 mL/hr) and the high urine output threshold (of, e.g., 625 mL/hr), the one or more outputs can include a prompt to increase the diuretic dosage rate and/or escalate the fluid therapy. In some embodiments, when the indicated urine sodium concentration is equal to or greater than the high urine sodium concentration threshold and the urine output is between the low urine output threshold and the high urine output threshold, the one or more outputs include the prompt to increase the diuretic dosage rate and/or escalate the fluid therapy only if the urine output is decreasing.

FIG. 7 is a block diagram illustrating a method 700 of providing outputs associated with a patient's fluid therapy based at least partially on a urine output (e.g., a urine output rate) and a urine oxygen content (e.g., a partial pressure of oxygen) of the patient, in accordance with embodiments of the present technology. The method 700 is illustrated as a series of steps, acts, processes, process portions, and/or blocks 702-706. At least some of the blocks 702-706 can be performed by a fluid management system and/or one or more components thereof, such as the system 100 and/or the controller 140 of FIG. 1, and/or the system 500 and/or the controller 508 of FIG. 5A.

At block 702, the method 700 includes obtaining a urine output of the patient. In some embodiments, obtaining the urine output of the patient includes obtaining a urine flow or output rate of the patient. Block 702 can be at least generally similar or identical to block 602 of the method 600 (FIG. 5).

At block 704, the method 700 includes obtaining a urine oxygen content of the patient. The urine oxygen content (e.g., a urine oxygen partial pressure) of excreted urine can be an important measure of health of a heart failure patient undergoing decongestion. For example, high urine oxygen content can indicate good and/or expected kidney function. Low urine oxygen content can be a sign of renal hypoxia, which can be an indicator (i) of poor kidney health, (ii) that the kidney may be consuming oxygen to actively retain fluid, and/or (iii) of acute kidney injury (AKI). Continuous measurement of urine oxygen concentration has the potential to be a much more rapid indicator of kidney health and/or the onset of acute kidney injury than, e.g., the measurement of serum creatinine, the current standard for measurement of AKI. The combination of urine oxygenation with obtained urine output (block 706) can provide even more diagnostic value. For example, data from a urine oxygen content sensor can be used to adjust a patient's fluid therapy if, e.g., the measured urine oxygen content falls outside of a predetermined range. The urine oxygen content can be obtained via a device that measures urinary oxygen partial pressure with an optical oxygen sensor that uses dynamic luminescence quenching, such as an optical oxygen sensor manufactured by PreSens Precision Sensing GmbH, headquartered in Regensburg, Germany. In some embodiments the oxygen sensor can be positioned upstream from the fluid therapy system and/or at least proximate to the patient's bladder, kidneys, and/or the patient's catheter. For example, the oxygen sensor can be positioned immediately adjacent to the patient's catheter or at least partially within the patient's bladder. Positioning the oxygen sensor at least proximate to (e.g., within 10 cm, 5 cm, 1 cm, 0.1 cm, etc. of) the patient's bladder, kidneys, and/or catheter is expected to improve the accuracy of the oxygen sensor's measurements. In some embodiments, the tubing used to provide urine from the patient to a fluid therapy system is permeable to oxygen, so positioning the oxygen sensor at least proximate to the patient's bladder, kidneys, and/or catheter can allow the oxygen sensor to obtain measurements before, or at least substantially before, oxygen can diffuse from the tubing. Positioning the urine oxygen sensor further away from the patient's bladder, kidneys, and/or catheter, is expected to increase the amount of oxygen that diffuse through the tubing before it can be measured, and especially so for patients with low urine rates. Urine oxygen content sensors are often delivered in an unsealed condition, e.g., exposed to environment air. Accordingly, block 704 may include waiting a predetermined amount of time (e.g., at least 5 minutes, 10 minutes, 15 minutes, 30 minutes, etc.) to allow the oxygen content sensor to equilibrate to the conditions within the system (e.g., the comparatively reduced oxygen content in the fluid line 119). In some embodiments, obtaining the urine oxygen content can include obtaining a temperature of urine using, e.g., the temperature sensor 418 (FIG. 4) and/or another temperature sensor. In at least some embodiments, the controller 140 can use temperature data from the temperature sensor to compensate for temperature-related effects on urine oxygen content data from the urine oxygen content sensor(s).

In some embodiments, obtaining the urine oxygen content (block 704) includes obtaining a blood oxygen saturation level of the patient. Generally, the interpretation of a urine oxygen sensor reading may vary based at least partially on the patient's blood oxygen saturation level. If the patient has a normal blood oxygen saturation level (e.g., 92-100% saturation), the urine oxygen sensor signal above can be interpreted as described herein. If, however, the patient is hypoxic, (e.g., blood oxygen saturation below 92%), this would also reduce the urine oxygen level. Accordingly, based on the patient's blood oxygen saturation level, the fluid therapy system (e.g., the controller 140) may use different urine oxygen content ranges to interpret the urine oxygen content signal and provide therapy modifications and/or other outputs. The fluid therapy system may also adjust the therapy based on the overall hypoxic state of the patient.

In some embodiments, obtaining the urine oxygen content (block 704) includes obtaining the presence of air in the fluid line 119 using, e.g., an ultrasonic sensor and/or another suitable sensor. For example, sudden changes in the urine oxygen content (greater than, e.g., 50%, 60%, 70%, 80%, 90%, 100%, etc.) can indicate the presence of air and/or other gas(es) in the fluid line. In such embodiments, the fluid therapy may be temporarily paused to purge the gas bubble(s) from the fluid line 119.

In some embodiments, prior to obtaining the urine oxygen content, the method 700 includes calibrating the one or more urine oxygen sensors. For example, in some embodiments fluid having a known oxygen content (e.g., saline solution with a known or zero oxygen content) is used to prime and/or flush the fluid line 119 and/or any sensors fluidly coupled thereto. Because the fluid has a known oxygen content, readings from the one or more urine oxygen sensor can be calibrated during the flushing stage.

In block 706, the method 700 includes providing one or more outputs associated with a fluid therapy received by the patient. Generally, the one or more outputs can include alerts or notifications provided to, e.g., a user or practitioner, and/or one or more modifications or adjustments to the patient's fluid therapy. One or more of the outputs can be based at least partially on the urine output (block 702) and/or a change thereto, and/or the urine oxygen content (block 704) and/or a change thereto (accounting for, e.g., the blood oxygen saturation level of the patient). For example, if urine oxygen content drops, the fluid therapy system may suggest one or more modifications to the patient's fluid therapy to maintain safety and/or prevent harm to the patient. Although described below as providing suggestions, those of ordinary skill in the art will appreciate that, in at least some embodiments, the one or more outputs can cause the system to automatically modify the therapy as described in the suggestion to adjust or optimize the therapy, such as by modifying the level of hydration fluid matching of urine output, modifying the diuretic dosage rate, etc.

More specifically, if the urine oxygen content is less than or equal to a low urine oxygen content threshold (e.g., up to 50 mmHg, 40 mmHg, 30 mmHg, 25 mmHg, 20 mmHg, 10 mmHg, 5 mmHg, etc., and/or decreases by at least 5%, 10%, 15%, 20%, 30%, etc., over a predetermined time period of, e.g., at least 15 minutes, 30 minutes, 1 hour, 2 hours, 6 hours, 12 hours, 24 hours, etc.) and the urine output is less than or equal to the low urine output threshold (of, e.g., 325 mL/hr), the one or more outputs can include a prompt to reduce the diuretic dosage rate, increase the saline matching, and/or stop the fluid therapy. If this state continues for an extended period of time with no user response, the system could automatically stop therapy.

If the urine oxygen content is less than or equal to a low urine oxygen content threshold and the urine output is equal to or greater than the high urine output threshold (e.g., 625 mL/hr), this suggests that the patient may be experiencing the polyuria phase of an acute kidney injury. Accordingly, increasing saline matching should be considered and the system can advise the user to increase saline matching, or increased saline matching could be started automatically. If the urine oxygen content is less than or equal to the low urine oxygen content threshold while the urine output rate is decreasing and/or between the high and low urine output thresholds, this suggests that the kidney is at high-risk of developing injury. When this state is detected, the system can suggest to the user or automatically increase fluid matching in order to attempt to improve renal function and/or renal perfusion before the kidney develops significant injury. Accordingly, the one or more outputs puts can include a prompt to reduce the diuretic dosage rate, increase saline matching, and/or stop therapy.

If the urine oxygen content is less than or equal to the low urine oxygen content threshold and the urine output is between the low urine output threshold (of, e.g., 325 mL/hr) and the high urine output threshold (of, e.g., 625 mL/hr), the one or more outputs can include a prompt to increase saline matching.

If the urine oxygen content is greater than or equal to a high urine oxygen content threshold (e.g., at least 20 mmHg, 25 mmHg, 50 mmHg, 60 mmHg, 40 mmHg, 80 mmHg, 90 mmHg, 100 mmHg, etc., and/or increases by at least than 5%, 10%, 15%, 20%, 30%, etc. over a predetermined time period of, e.g., at least 15 minutes, 30 minutes, 1 hour, 2 hours, 6 hours, 12 hours, 24 hours, etc.) and the urine output is less than or equal to the low urine output threshold (of, e.g., 325 mL/hr), it suggests that the kidney can tolerate increased diuretic infusion—this could allow the acceleration of recommendations to “re-ramp” the diuretic infusion, or to add a second diuretic if the max continuous initial diuretic dose has been reached. Accordingly, the one or more outputs can include a prompt to escalate therapy by, e.g., increasing the diuretic delivery rate, administering an additional diuretic (e.g., thiazide), or adjusting (e.g., increasing) the hydration fluid matching.

If the urine oxygen content is greater than or equal to the high urine oxygen content threshold and the urine output is equal to or greater than the high urine output threshold (of, e.g., 625 mL/hr), this suggests that the kidney can tolerate a reduction in the saline infusion, which would increase net fluid removal and net sodium removal. Accordingly, the one or more outputs can include a prompt to reduce the hydration fluid infusion rate.

If the urine oxygen content is greater than or equal to the high urine oxygen content threshold and the urine output rate is the urine output is between the low urine output threshold (of, e.g., 325 mL/hr) and the high urine output threshold (of, e.g., 625 mL/hr) and/or has a slope that is trending toward the low urine output threshold, the one or more outputs can include a recommendation to “re-ramp” the patient's therapy, and/or to administer an additional diuretic (if, e.g., the max continuous dose of the first diuretic (e.g., furosemide) has been reached). Accordingly, the one or more outputs can include a prompt to increase the diuretic dosage rate, administer a second diuretic, and/or reduce the hydration fluid infusion rate.

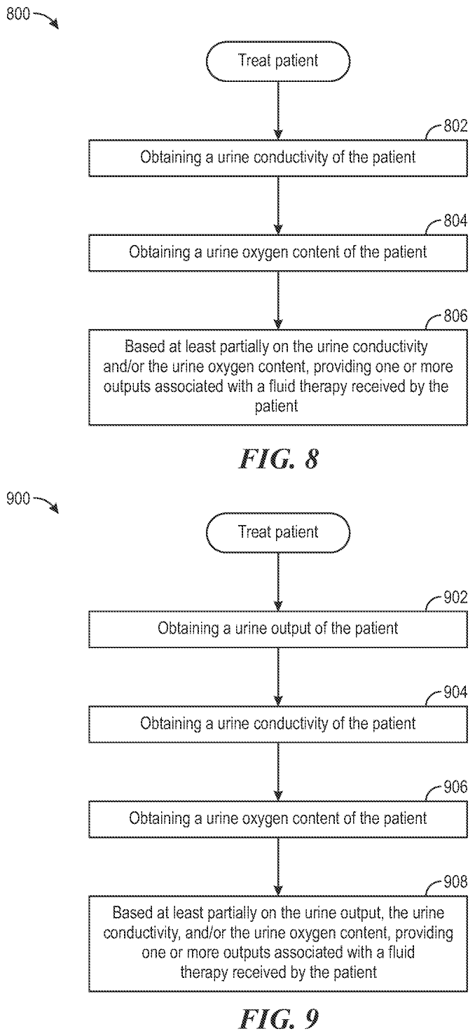

FIG. 8 is a block diagram illustrating a method 800 of providing outputs associated with a patient's fluid therapy based at least partially on a urine conductivity and a urine oxygen content (e.g., a partial pressure of oxygen in urine) of the patient, in accordance with embodiments of the present technology. The method 800 is illustrated as a series of steps, acts, processes, process portions, and/or blocks 802-806. At least some of the blocks 802-806 can be performed by a fluid management system and/or one or more components thereof, such as the system 100 and/or the controller 140 of FIG. 1, and/or the system 500 and/or the controller 508 of FIG. 5A.

At block 802, the method 800 includes obtaining a urine conductivity of the patient. At least some aspects of block 802 can be at least generally similar or identical to block 604 of the method 600 (FIG. 6).

At block 804, the method 800 includes obtaining a urine oxygen content of the patient. At least some aspects of block 804 can be at least generally similar or identical to block 704 of the method 700 (FIG. 7).

At block 806, the method 800 includes providing one or more outputs associated with a fluid therapy received by the patient. Generally, the one or more outputs can include one or more alerts or notifications provided to, e.g., a user or practitioner, and/or one or more modifications or adjustments to the patient's fluid therapy. One or more of the outputs can be based at least partially on the urine conductivity (block 802) and/or the urine oxygen content (block 804). For example, the fluid therapy system may suggest one or more modifications to the patient's fluid therapy to, e.g., adjust or optimize treatment, maintain safety, and/or prevent harm to the patient based at least partially on, e.g., the urine conductivity and/or a change thereto, and/or the urine oxygen content and/or a change thereto. Although described below as providing suggestions, those of ordinary skill in the art will appreciate that, in at least some embodiments, the one or more outputs can cause the system to automatically modify the therapy as described in the suggestion to adjust or optimize the therapy, such as by modifying the level of hydration fluid matching of urine output, modifying the diuretic dosage rate, etc.

In operation, if the urine oxygen content is less than or equal to the low urine oxygen content threshold and an indication of the urine sodium concentration (e.g., the urine conductivity) corresponds to a urine sodium concentration that is less than or equal to the low urine sodium concentration threshold (of, e.g., 75 mmol/L), this may indicate that the patient's kidneys are in a potentially unhealthy state. Accordingly, the one or more outputs can include suggesting an adjustment (e.g., decrease) to the diuretic dosage rate, an increase to the hydration fluid matching, and/or that the therapy should be stopped. If the urine oxygen content is less than or equal to the low urine oxygen content threshold and the urine conductivity corresponds to a urine sodium concentration that is equal to or greater than the high urine sodium concentration threshold (of, e.g., 110 mmol/L), this, too, can indicate that the patient's kidneys are in a potentially unhealthy state. Accordingly, the one or more outputs can include suggesting a decrease to the diuretic dosage rate, an increase to the hydration fluid matching, and/or that the therapy should be stopped.

Additionally or alternatively, if the urine oxygen content is less than or equal to the low urine oxygen content threshold and the urine conductivity corresponds to a urine sodium concentration that is between the low urine sodium concentration threshold (of, e.g., 75 mmol/L) and the high urine sodium concentration threshold (of, e.g., 100 mmol/L) and/or has a slope trending toward the low urine sodium concentration threshold, the one or more outputs can include a suggestion to increase the hydration fluid matching. If, on the other hand, the urine oxygen concentration is high, this can indicate that the patient's kidneys are healthy, as described previously herein (with reference to, e.g., FIG. 7). More specifically, if the urine oxygen content is greater than or equal to the high urine oxygen content threshold and the urine conductivity corresponds to a urine sodium concentration that is less than or equal to the low urine sodium concentration threshold (of, e.g., 75 mmol/L), this can indicate that the kidneys are healthy but that the patient has low urine output. Accordingly, the one or more inputs can include suggesting an increase to the diuretic dosage rate, suggesting administration of a second diuretic (e.g., a thiazide), and/or an adjustment (e.g., an increase) to the hydration fluid matching.

If the urine oxygen content is greater than or equal to the high urine oxygen content threshold and the urine conductivity corresponds to a urine sodium concentration that is equal to or greater than the high urine sodium concentration threshold (of, e.g., 100 mmol/L), the fluid therapy system may not make changes to the diuretic dosage rate. In some embodiments, the one or more outputs can include a suggestion to continue administering the diuretic at the diuretic dosage rate and/or the reduce to the hydration fluid infusion rate.

If the urine oxygen content is greater than or equal to the high urine oxygen content threshold and the urine conductivity corresponds to a urine sodium concentration that is between the low urine sodium concentration threshold (of, e.g., 75 mmol/L) and the high urine sodium concentration threshold (of, e.g., 100 mmol/L) and/or has a slope trending toward the low urine sodium concentration threshold, the one or more outputs can include a suggestion to continue therapy. If a urine output of the patient is decreasing, the one or more outputs can include a suggestion to increase the diuretic dosage rate.

FIG. 9 is a block diagram illustrating a method 900 of providing outputs associated with a patient's fluid therapy based at least partially on a urine output (e.g., a urine output rate), a urine conductivity, and a urine oxygen content (e.g., a partial pressure of oxygen in urine) of the patient, in accordance with embodiments of the present technology. The method 900 is illustrated as a series of steps, acts, processes, process portions, and/or blocks 902-908. At least some of the blocks 902-908 can be performed by a fluid management system and/or one or more components thereof, such as the system 100 and/or the controller 140 of FIG. 1, and/or the system 500 and/or the controller 508 of FIG. 5A.

At block 902, the method 900 includes obtaining a urine output (e.g., a urine output rate) of the patient. At least some aspects of block 902 can be at least generally similar or identical to block 602 of the method 600 (FIG. 6).

At block 904, the method 900 includes obtaining a urine conductivity of the patient. At least some aspects of block 904 can be at least generally similar or identical to block 604 of the method 600 (FIG. 6).

At block 906, the method 800 includes obtaining a urine oxygen content of the patient. At least some aspects of block 906 can be at least generally similar or identical to block 704 of the method 700 (FIG. 7).

At block 908, the method 900 includes providing one or more outputs associated with a fluid therapy received by the patient. Generally, the one or more outputs can include one or more alerts or notifications provided to, e.g., a user or practitioner, and/or one or more modifications or adjustments to the patient's fluid therapy. One or more of the outputs can be based at least partially on the urine output (block 902), the urine conductivity (block 904), and/or the urine oxygen content (block 906). For example, the fluid therapy system may suggest one or more modifications to the patient's fluid therapy to, e.g., adjust or optimize treatment, maintain safety, and/or prevent harm to the patient based at least partially on, e.g., the urine output and/or a change thereto, the urine conductivity and/or a change thereto, and/or urine oxygen content and/or a change thereto. Although described below as providing suggestions, those of ordinary skill in the art will appreciate that, in at least some embodiments, the one or more outputs can cause the system to automatically modify the therapy as described in the suggestion to adjust or optimize the therapy, such as by modifying the level of hydration fluid matching of urine output, modifying the diuretic dosage rate, etc.

In operation, if the urine conductivity corresponds to a urine sodium concentration that is less than or equal to the low urine sodium concentration threshold (of, e.g., 75 mmol/L), the urine output is less than or equal to the low urine output threshold (of, e.g., 325 mL/hr), and the urine oxygen content is less than or equal to the low urine oxygen content threshold, the one or more outputs can include a suggestion to stop therapy and/or to increase hydration fluid matching. If the urine conductivity corresponds to a urine sodium concentration that is less than or equal to the low urine sodium concentration threshold (of, e.g., 75 mmol/L), the urine output is less than or equal to the low urine output threshold (of, e.g., 325 mL/hr), and the urine oxygen content is greater than or equal to the high urine oxygen content threshold, the one or more outputs can include a suggestion to increase the diuretic dosage rate and/or to administer a second diuretic (e.g., a thiazide).

If the urine conductivity corresponds to a urine sodium concentration that is less than or equal to the low urine sodium concentration threshold (of, e.g., 75 mmol/L), the urine output is equal to or greater than the high urine output threshold (of, e.g., 625 mL/hr), and the urine oxygen content is less than or equal to the low urine oxygen content threshold, the one or more outputs can include a suggestion to increase electrolyte monitoring, to reduce hydration fluid matching, to increase the diuretic dosage rate, to stop the therapy, and/or block 908 can include providing an alert to, e.g., a user regarding the patient's status. If the urine conductivity corresponds to a urine sodium concentration that is less than or equal to the low urine sodium concentration threshold (of, e.g., 75 mmol/L), the urine output is equal to or greater than the high urine output threshold (of, e.g., 625 mL/hr), and the urine oxygen content is greater than or equal to the high urine oxygen content threshold, the one or more outputs can include a suggestion to increase electrolyte monitoring.

If the urine conductivity corresponds to a urine sodium concentration that is less than or equal to the low urine sodium concentration threshold (of, e.g., 75 mmol/L), the urine output is between the low urine output threshold (of, e.g., 325 mL/hr) and the high urine output threshold (of, e.g., 625 mL/hr), and the urine oxygen content is less than or equal to the low urine oxygen content threshold, the one or more outputs can include a suggestion to increase electrolyte monitoring. If the urine conductivity corresponds to a urine sodium concentration that is less than or equal to the low urine sodium concentration threshold (of, e.g., 75 mmol/L), the urine output is between the low urine output threshold (of, e.g., 325 mL/hr) and the high urine output threshold (of, e.g., 625 mL/hr), and the urine oxygen content is greater than or equal to the high urine oxygen content threshold, the one or more outputs can include a suggestion to increase the diuretic dosage rate and/or to administer an additional diuretic.

If the urine conductivity corresponds to a urine sodium concentration that is equal to or greater than the high urine sodium concentration threshold (of, e.g., 100 mmol/L), the urine output is less than or equal to the low urine output threshold (of, e.g., 325 mL/hr), and the urine oxygen content is less than or equal to the low urine oxygen content threshold, the one or more outputs can include a suggestion to adjust (e.g., increase) the diuretic dosage rate and/or stop therapy. If the urine conductivity corresponds to a urine sodium concentration that is less than or equal to the low urine sodium concentration threshold (of, e.g., 75 mmol/L), the urine output is less than or equal to the low urine output threshold (of, e.g., 325 mL/hr), and the urine oxygen content is greater than or equal to the high urine oxygen content threshold, the one or more outputs can include a suggestion to increase the diuretic dosage rate and/or administer an additional diuretic.

If the urine conductivity corresponds to a urine sodium concentration that is equal to or greater than the high urine sodium concentration threshold (of, e.g., 100 mmol/L), the urine output is equal to or greater than the high urine output threshold (of, e.g., 625 mL/hr), and the urine oxygen content is less than or equal to the low urine oxygen content threshold, the one or more outputs can include a suggestion to maintain the current therapy (e.g., no changes), and/or a suggestion to increase the hydration fluid to matching to, e.g., reduce or prevent sodium avidity and/or renal damage. If the urine conductivity corresponds to a urine sodium concentration that is equal to or greater than the high urine sodium concentration threshold (of, e.g., 100 mmol/L), the urine output is equal to or greater than the high urine output threshold (of, e.g., 625 mL/hr), and the urine oxygen content is greater than or equal to the high urine oxygen content threshold, the one or more outputs can include a suggestion to maintain the current therapy (e.g., no changes), and/or a suggestion to decrease the hydration fluid to matching to, e.g., increase or maximize removal of fluid and/or salt.

If the urine conductivity corresponds to a urine sodium concentration that is equal to or greater than the high urine sodium concentration threshold (of, e.g., 100 mmol/L), the urine output is between the low urine output threshold (of, e.g., 325 mL/hr) and the high urine output threshold (of, e.g., 625 mL/hr), and the urine oxygen content is less than or equal to the low urine oxygen content threshold, the one or more outputs can include a suggestion to increase the hydration fluid matching. If the urine conductivity corresponds to a urine sodium concentration that is equal to or greater than the high urine sodium concentration threshold (of, e.g., 100 mmol/L), the urine output is between the low urine output threshold (of, e.g., 325 mL/hr) and the high urine output threshold (of, e.g., 625 mL/hr), and the urine oxygen content is greater than or equal to the high urine oxygen content threshold, the one or more outputs can include a suggestion to increase the diuretic dosage rate. In some embodiments, the suggestion to increase the diuretic dosage rate can be in response to a decrease in the urine flow rate great than a urine flow rate decrease threshold.

### III. EXAMPLES

Additional aspects of various embodiments of the present technology are described with reference to the following examples: 1. A method for providing fluid therapy, the method comprising:
obtaining an output rate of urine from a patient;
causing a diuretic to be provided to the patient at a dosage rate;
causing a hydration fluid to be provided to the patient at a hydration rate;
obtaining one of more characteristics of the urine, wherein the one or more characteristics comprises urine conductivity and/or urine oxygen content; and
based on the obtained characteristics of the urine, providing an output associated with adjusting at least one of the dosage rate or the hydration rate.

2. The method of any one of the clauses herein, wherein the urine conductivity corresponds to a determined urine sodium concentration, and wherein, when the determined urine sodium concentration is below a predetermined low urine sodium threshold and the urine output rate is below a predetermined low urine output rate, providing the output comprises providing the output to stop therapy, to increase the dosage rate, and/or increase the hydration rate.

3. The method of any one of the clauses herein, wherein the urine conductivity corresponds to a determined urine sodium concentration, and wherein, when the determined urine sodium concentration is above a predetermined high urine sodium threshold and the urine output rate is below a predetermined low urine output rate, providing the output comprises providing the output to increase the hydration rate, decrease the diuretic dosage rate, and/or stop therapy.

4. The method of any one of the clauses herein, wherein the urine conductivity corresponds to a determined urine sodium concentration, and wherein, when the determined urine sodium concentration is below a predetermined low urine sodium threshold and the urine output rate is above a predetermined high urine output rate, providing the output comprises providing the output to increase the dosage rate and/or reduce the hydration rate.

5. The method of any one of the clauses herein, wherein the urine conductivity corresponds to a determined urine sodium concentration, and wherein, when the determined urine sodium concentration is above a predetermined high urine sodium threshold and the urine output rate is above a predetermined high urine output rate, providing the output comprises providing the output to decrease the hydration rate.

6. The method of any one of the clauses herein, wherein the urine conductivity corresponds to a determined urine sodium concentration, and wherein, when the determined urine sodium concentration is above a predetermined high urine sodium threshold and the urine output rate is decreasing, providing the output comprises providing the output to increase the dosage rate and/or increase the hydration rate and/or administer an additional diuretic.

7. The method of any one of the clauses herein, wherein the predetermined high urine sodium threshold is 100 millimoles (mmol)/Liter (L) and the predetermined low urine sodium threshold is 75 mmol/L.

8. The method of any one of the clauses herein, wherein the predetermined low urine output rate is 325 milliliters (mL)/hour (hr) and the predetermined high urine output threshold is 625 mL/hr.

9. The method of any one of the clauses herein, wherein the one or more characteristics comprises urine sodium concentration.

10. The method of any one of the clauses herein, wherein the urine conductivity corresponds to a determined urine sodium concentration, and wherein obtaining one of more characteristics of the urine comprises obtaining a rate of change of the determined urine sodium concentration.

11. The method of any one of the clauses herein, wherein adjusting at least one of the dosage rate or the hydration rate is further based on the urine output rate.

12. The method of any one of the clauses herein, wherein, when the urine oxygen content is below a predetermined low urine oxygen threshold and the urine output rate is below a predetermined low urine output rate, providing the output comprises providing the output to decrease the dosage rate, increase the hydration rate, and/or stop therapy.

13. The method of any one of the clauses herein, wherein, when the urine oxygen content is above a predetermined high urine oxygen threshold and the urine output rate is below a predetermined low urine output rate, providing the output comprises providing the output to increase the hydration rate, increase the diuretic dosage rate, and/or administer an additional diuretic.

14. The method of any one of the clauses herein, wherein, when the urine oxygen content is below a predetermined low urine oxygen threshold and the urine output rate is above a predetermined high urine output rate, providing the output comprises providing the output to decrease the dosage rate and/or increase the hydration rate.

15. The method of any one of the clauses herein, wherein, when the urine oxygen content is above a predetermined high urine oxygen threshold and the urine output rate is decreasing, providing the output comprises providing the output to increase the dosage rate.

16. The method of any one of the clauses herein, wherein, when the urine oxygen content is below a predetermined low urine oxygen threshold and the urine output rate is decreasing, providing the output comprises providing the output to increase the dosage rate and/or increase the hydration rate.

17. The method of any one of the clauses herein, wherein the predetermined high urine oxygen threshold is 25 mmHg and the predetermined low urine oxygenation threshold is 10 mmHg.

18. The method of any one of the clauses herein, wherein the urine conductivity corresponds to a determined urine sodium concentration, and wherein, when the determined urine sodium concentration is below a predetermined low urine sodium threshold and the urine oxygen content is below a predetermined low urine oxygen threshold, providing the output comprises providing the output to decrease the dosage rate and/or increase the hydration rate.

19. The method of any one of the clauses herein, wherein the urine conductivity corresponds to a determined urine sodium concentration, and wherein, when the determined urine sodium concentration is above a predetermined high urine sodium threshold and the urine oxygen content is below a predetermined low urine oxygen threshold, providing the output comprises providing the output to increase the hydration rate and/or decrease the diuretic dosage rate.

20. The method of any one of the clauses herein, wherein the urine conductivity corresponds to a determined urine sodium concentration, and wherein, when the determined urine sodium concentration is below a predetermined low urine sodium threshold and the urine oxygen content is above a predetermined high urine oxygen threshold, providing the output comprises providing the output to increase the dosage rate. 21. The method of any one of the clauses herein, wherein the urine conductivity corresponds to a determined urine sodium concentration, and wherein, when the determined urine sodium concentration is above a predetermined high urine sodium threshold and the urine oxygen content is above a predetermined high urine oxygen threshold, providing the output comprises providing the output to decrease the hydration rate. 22. The method of any one of the clauses herein, wherein the urine conductivity corresponds to a determined urine sodium concentration, and wherein, when the urine oxygen content is above a predetermined high urine oxygen threshold and the urine output rate is decreasing, providing the output comprises providing the output to increase the dosage rate. 23. A fluid therapy system, comprising:
a urine measurement device configured to measure urine output from a patient;
a first pump configured to provide a diuretic to the patient at a diuretic dosage rate;
a second pump configured to provide a hydration fluid to the patient at a hydration fluid infusion rate;
a plurality of sensors configured to measure characteristics of the urine from the patient, the sensors including a urine conductivity sensor and/or a urine oxygen sensor;
one or more processors; and
tangible, non-transitory computer-readable media having instructions that, when executed by the one or more processors, cause the fluid therapy system to perform operations comprising—
   obtaining, via the urine measurement device, an output rate of urine from a patient;
   causing, via the first pump, a diuretic to be provided to the patient at a dosage rate;
   causing, via the second pump, a hydration fluid to be provided to the patient at a hydration rate;
   obtaining, via the sensors, one of more characteristics of the urine, wherein the one or more characteristics comprises urine conductivity and/or urine oxygen content; and
   based on the obtained characteristics of the urine, providing an output associated with adjusting at least one of the dosage rate or the hydration rate.

24. The fluid therapy system of any one of the clauses herein, wherein the urine conductivity corresponds to a determined urine sodium concentration, and wherein, when the determined urine sodium concentration is below a predetermined low urine sodium threshold and the urine output rate is below a predetermined low urine output rate, providing the output comprises providing the output to stop therapy, to increase the dosage rate, and/or increase the hydration rate.

25. The fluid therapy system of any one of the clauses herein, wherein the urine conductivity corresponds to a determined urine sodium concentration, and wherein, when the determined urine sodium concentration is above a predetermined high urine sodium threshold and the urine output rate is below a predetermined low urine output rate, providing the output comprises providing the output to increase the hydration rate, decrease the diuretic dosage rate, and/or stop therapy.

26. The fluid therapy system of any one of the clauses herein, wherein the urine conductivity corresponds to a determined urine sodium concentration, and wherein, when the determined urine sodium concentration is below a predetermined low urine sodium threshold and the urine output rate is above a predetermined high urine output rate, providing the output comprises providing the output to increase the dosage rate and/or reduce the hydration rate.

27. The fluid therapy system of any one of the clauses herein, wherein the urine conductivity corresponds to a determined urine sodium concentration, and wherein, when the determined urine sodium concentration is above a predetermined high urine sodium threshold and the urine output rate is above a predetermined high urine output rate, providing the output comprises providing the output to decrease the hydration rate.

28. The fluid therapy system of any one of the clauses herein, wherein the urine conductivity corresponds to a determined urine sodium concentration, and wherein, when the determined urine sodium concentration is above a predetermined high urine sodium threshold and the urine output rate is decreasing, providing the output comprises providing the output to increase the dosage rate and/or increase the hydration rate and/or administer an additional diuretic.

29. The fluid therapy system of any one of the clauses herein, wherein the predetermined high urine sodium threshold is 100 millimoles (mmol)/Liter (L) and the predetermined low urine sodium threshold is 75 mmol/L.

30. The fluid therapy system of any one of the clauses herein, wherein the predetermined low urine output rate is 325 milliliters (mL)/hour (hr) and the predetermined high urine output threshold is 625 mL/hr.

31. The fluid therapy system of any one of the clauses herein, wherein the one or more characteristics comprises urine sodium concentration.

32. The fluid therapy system of any one of the clauses herein, wherein the urine conductivity corresponds to a determined urine sodium concentration, and wherein obtaining one of more characteristics of the urine comprises obtaining a rate of change of the determined urine sodium concentration.

33. The fluid therapy system of any one of the clauses herein, wherein adjusting at least one of the dosage rate or the hydration rate is further based on the urine output rate.

34. The fluid therapy system of any one of the clauses herein, wherein, when the urine oxygen content is below a predetermined low urine oxygen threshold and the urine output rate is below a predetermined low urine output rate, providing the output comprises providing the output to decrease the dosage rate, increase the hydration rate, and/or stop therapy.

35. The fluid therapy system of any one of the clauses herein, wherein, when the urine oxygen content is above a predetermined high urine oxygen threshold and the urine output rate is below a predetermined low urine output rate, providing the output comprises providing the output to increase the hydration rate, increase the diuretic dosage rate, and/or administer an additional diuretic.

36. The fluid therapy system of any one of the clauses herein, wherein, when the urine oxygen content is below a predetermined low urine oxygen threshold and the urine output rate is above a predetermined high urine output rate, providing the output comprises providing the output to decrease the dosage rate and/or increase the hydration rate.

37. The fluid therapy system of any one of the clauses herein, wherein, when the urine oxygen content is above a predetermined high urine oxygen threshold and the urine output rate is decreasing, providing the output comprises providing the output to increase the dosage rate.

38. The fluid therapy system of any one of the clauses herein, wherein, when the urine oxygen content is below a predetermined low urine oxygen threshold and the urine output rate is decreasing, providing the output comprises providing the output to increase the dosage rate and/or increase the hydration rate.

39. The fluid therapy system of any one of the clauses herein, wherein the predetermined high urine oxygen threshold is 25 mmHg and the predetermined low urine oxygenation threshold is 10 mmHg.

40. The fluid therapy system of any one of the clauses herein, wherein the urine conductivity corresponds to a determined urine sodium concentration, and wherein, when the determined urine sodium concentration is below a predetermined low urine sodium threshold and the urine oxygen content is below a predetermined low urine oxygen threshold, providing the output comprises providing the output to decrease the dosage rate and/or increase the hydration rate.

41. The fluid therapy system of any one of the clauses herein, wherein the urine conductivity corresponds to a determined urine sodium concentration, and wherein, when the determined urine sodium concentration is above a predetermined high urine sodium threshold and the urine oxygen content is below a predetermined low urine oxygen threshold, providing the output comprises providing the output to increase the hydration rate and/or decrease the diuretic dosage rate.

42. The fluid therapy system of any one of the clauses herein, wherein the urine conductivity corresponds to a determined urine sodium concentration, and wherein, when the determined urine sodium concentration is below a predetermined low urine sodium threshold and the urine oxygen content is above a predetermined high urine oxygen threshold, providing the output comprises providing the output to increase the dosage rate.

43. The fluid therapy system of any one of the clauses herein, wherein the urine conductivity corresponds to a determined urine sodium concentration, and wherein, when the determined urine sodium concentration is above a predetermined high urine sodium threshold and the urine oxygen content is above a predetermined high urine oxygen threshold, providing the output comprises providing the output to decrease the hydration rate.

44. The fluid therapy system of any one of the clauses herein, wherein the urine conductivity corresponds to a determined urine sodium concentration, and wherein, when the urine oxygen content is above a predetermined high urine oxygen threshold and the urine output rate is decreasing, providing the output comprises providing the output to increase the dosage rate.

45. A urine cartridge for use with a fluid therapy system, the urine cartridge comprising:
a sensor assembly, including—
   a housing that defines a fluid conduit, wherein the fluid conduit has a first end and a second end positioned above the first end, and wherein the fluid conduit is configured to receive urine from a patient at the first end and direct the received urine into a fluid line at the second end,
   a pair of electrically conductive contacts defining a conductivity sensor configured to measure a conductivity of fluid within the fluid conduit, and
   a temperature sensor configured to measure a temperature of fluid within the fluid conduit; and
a body carrying the sensor assembly and configured to operably engage the sensor assembly and the fluid line with the fluid therapy system.

46. The urine cartridge of any one of the clauses herein, wherein the housing of the sensor assembly further defines a plurality of sensing apertures, wherein the temperature sensor and the pair of electrically conductive contacts are positioned at least partially within the fluid conduit via a respective one of the plurality of sensing apertures.

47. The urine cartridge of any one of the clauses herein, wherein the housing of the sensor assembly defines an interior, and wherein the conductivity sensor and/or the temperature sensor are positioned at least partially within the interior.

48. The urine cartridge of any one of the clauses herein, wherein the pair of electrically conductive contacts are spaced apart from one another by a distance of at least 0.44 inches.

49. The urine cartridge of any one of the clauses herein, wherein the body includes—
a urine line coupling feature configured to couple to a first portion of the fluid line to direct urine flow through the first portion in a first direction, and
a urine line return feature configured to couple to a second portion of the fluid line to direct urine flow through the second portion in a second direction opposite the first direction.

50. The urine cartridge of any one of the clauses herein, wherein the fluid conduit has a first inner diameter, and wherein the fluid line has a second inner diameter different than the first inner diameter.

51. The urine cartridge of any one of the clauses herein, wherein the fluid conduit has a first inner diameter, and wherein the fluid line has a second inner diameter less than the first inner diameter.

52. The urine cartridge of any one of the clauses herein, wherein the temperature sensor is positioned between the pair of electrically conductive contacts.

53. The urine cartridge of any one of the clauses herein, wherein the body includes—
a urine line coupling feature configured to couple to a first portion of the fluid line to direct urine flow through the first portion in a vertically upward direction, and
a urine line return feature configured to couple to a second portion of the fluid line to direct urine flow through the second portion in a vertically downward.

54. A fluid therapy system, comprising:
a proximal fluid line configured to receive urine from a patient,
a distal fluid line configured to direct the received urine toward a container;
a urine flow assembly including a sensor configured to generate sensor data based on the received urine; and
a urine cartridge including—
   a body configured to (i) couple to a portion of the distal fluid line, (ii) couple to the urine flow assembly, and (iii), when coupled to the urine flow assembly, operably engage the portion of the distal fluid line with the sensor, and
   a sensor assembly, including—
      a housing that defines a fluid conduit, wherein the fluid conduit is (i) coupled to the proximal fluid line to receive urine from the patient via the proximal fluid line and (ii) coupled to the distal fluid line and configured to direct the received urine toward the container via the distal fluid line,
      a pair of electrically conductive contacts defining a conductivity sensor configured to measure a conductivity of fluid within the fluid conduit, and
      a temperature sensor positioned generally adjacent to the pair of electrically conductive contacts and configured to measure a temperature of fluid within the fluid conduit.

55. The fluid therapy system of any one of the clauses herein, wherein the sensor defines a slot, and wherein the body of the urine cartridge is configured to position the portion of the distal fluid line within the slot when coupled to the urine flow assembly.

56. The fluid therapy system of any one of the clauses herein, wherein the urine flow assembly includes a receiving feature, and wherein the body of the urine cartridge includes a couple feature configured to releasably engage the receiving feature to operably engage the portion of the distal fluid line with the sensor.

57. The fluid therapy system of any one of the clauses herein, wherein the portion of the distal fluid line is an upstream portion of the distal fluid line, and wherein the body of the urine cartridge defines a channel configured to receive a downstream portion of the distal fluid line.

58. The fluid therapy system of any one of the clauses herein, wherein the fluid conduit has a first inner diameter, and wherein the proximal fluid line and/or the distal fluid line have a second inner diameter less than the first inner diameter.

59. The fluid therapy system of any one of the clauses herein wherein the temperature sensor is positioned upstream or downstream from one or both of the pair of electrically conductive contacts.

60. The fluid therapy system of any one of the clauses herein, further comprising:
a first pump configured to provide a diuretic to the patient at a diuretic dosage rate;
a second pump configured to provide a hydration fluid to the patient at a hydration fluid infusion rate;
one or more processors; and
tangible, non-transitory computer-readable media having instructions that, when executed by the one or more processors, cause the fluid therapy system to perform operations comprising—
   obtaining, via the sensor, an output rate of urine from a patient;
   causing, via the first pump, a diuretic to be provided to the patient at a dosage rate;
   causing, via the second pump, a hydration fluid to be provided to the patient at a hydration rate;
   obtaining, via the conductivity sensor and/or the temperature sensor, one of more characteristics of the urine, wherein the one or more characteristics comprises urine conductivity; and
   based on the obtained characteristics of the urine, providing an output associated with adjusting at least one of the dosage rate or the hydration rate.

61. A method for providing fluid therapy, the method comprising:
causing, via a first pump, a diuretic to be provided to a patient at a diuretic dosage rate;
causing, via a second pump, a hydration fluid to be provided to the patient at a hydration rate;
receiving, at a first end of a fluid conduit of a sensor assembly, urine from a patient via a proximal fluid line;
obtaining, via a conductivity sensor and/or a temperature sensor of the sensor assembly, one of more characteristics of the urine within the fluid conduit;
directing the urine to flow out of a second end of the fluid conduit toward a container via a distal fluid line, the second end positioned above the first end; and
based on the obtained characteristics of the urine, providing an output associated with adjusting the diuretic dosage rate and/or the hydration rate.

62. The method of any one of the clauses herein, wherein directing the urine to flow out of the second end of the fluid conduit includes directing the urine to flow through an upstream portion of the distal fluid line in a first direction, and wherein the method further comprises directing the urine to flow through a downstream portion of the distal fluid line in a second direction opposite the first direction.

63. The method of any one of the clauses herein, further comprising receiving an upstream portion of the distal fluid line at least partially within one or more urine line coupling features of a urine cartridge including the sensor assembly.

64. The method of any one of the clauses herein, wherein the conductivity sensor includes a pair of electrically conductive contacts, and wherein obtaining the one or more characteristics includes obtaining, via the temperature sensor, a temperature of the urine between the pair of electrically conductive contacts.

65. The method of any one of the clauses herein, wherein adjusting the diuretic dosage rate and/or the hydration rate includes increasing or decreasing the diuretic dosage rate and/or the hydration rate.

66. The method of any one of the clauses herein, further comprising, prior to receiving the urine from the patient, flushing the sensor assembly with a solution of know electrolyte content to calibrate the conductivity sensor.

### IV. CONCLUSION

It will be apparent to those having skill in the art that changes may be made to the details of the above-described embodiments without departing from the underlying principles of the present technology. In some cases, well known structures and functions have not been shown or described in detail to avoid unnecessarily obscuring the description of the embodiments of the present technology. Although blocks of methods may be presented herein in a particular order, alternative embodiments may perform the blocks in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Throughout this disclosure, the singular terms “a,” “an,” and “the” include plural referents unless the context clearly indicates otherwise. The use of the term “and/or” in reference to a list of two or more items is to be interpreted as including (a) any single item in the list, (b) all of the items in the list, or (c) any combination of the items in the list. Additionally, the term “comprising,” “including,” and “having” should be interpreted to mean including at least the recited feature(s) such that any greater number of the same feature and/or additional types of other features are not precluded.

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing concentrations, pressures, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

The disclosure set forth above is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

I/we claim:

1. A urine cartridge for use with a fluid therapy system, the urine cartridge comprising:
a sensor assembly, including—
a housing that defines a fluid conduit, wherein the fluid conduit has a first end and a second end positioned above the first end, and wherein the fluid conduit is configured to receive urine from a patient at the first end and direct the received urine into a fluid line at the second end,
a pair of electrically conductive contacts defining a conductivity sensor configured to measure a conductivity of fluid within the fluid conduit, and
a temperature sensor configured to measure a temperature of fluid within the fluid conduit; and
a body carrying the sensor assembly and configured to operably engage the sensor assembly and the fluid line with the fluid therapy system.

2. The urine cartridge of claim 1, wherein the housing of the sensor assembly further defines a plurality of sensing apertures, wherein the temperature sensor and the pair of electrically conductive contacts are positioned at least partially within the fluid conduit via a respective one of the plurality of sensing apertures.

3. The urine cartridge of claim 1, wherein the housing of the sensor assembly defines an interior, and wherein the conductivity sensor and/or the temperature sensor are positioned at least partially within the interior.

4. The urine cartridge of claim 1, wherein the pair of electrically conductive contacts are spaced apart from one another by a distance of at least 0.44 inches.

5. The urine cartridge of claim 1, wherein the body includes—
a urine line coupling feature configured to couple to a first portion of the fluid line to direct urine flow through the first portion in a first direction, and
a urine line return feature configured to couple to a second portion of the fluid line to direct urine flow through the second portion in a second direction opposite the first direction.

6. The urine cartridge of claim 1, wherein the fluid conduit has a first inner diameter, and wherein the fluid line has a second inner diameter different than the first inner diameter.

7. The urine cartridge of claim 1, wherein the fluid conduit has a first inner diameter, and wherein the fluid line has a second inner diameter less than the first inner diameter.

8. The urine cartridge of claim 1, wherein the temperature sensor is positioned between the pair of electrically conductive contacts.

9. The urine cartridge of claim 1, wherein the body includes—
a urine line coupling feature configured to couple to a first portion of the fluid line to direct urine flow through the first portion in a vertically upward direction, and
a urine line return feature configured to couple to a second portion of the fluid line to direct urine flow through the second portion in a vertically downward.

10. A fluid therapy system, comprising:
a proximal fluid line configured to receive urine from a patient,
a distal fluid line configured to direct the received urine toward a container;
a urine flow assembly including a sensor configured to generate sensor data based on the received urine; and
a urine cartridge including—
a body configured to (i) couple to a portion of the distal fluid line, (ii) couple to the urine flow assembly, and (iii), when coupled to the urine flow assembly, operably engage the portion of the distal fluid line with the sensor, and
a sensor assembly, including—
a housing that defines a fluid conduit, wherein the fluid conduit is (i) coupled to the proximal fluid line to receive urine from the patient via the proximal fluid line and (ii) coupled to the distal fluid line and configured to direct the received urine toward the container via the distal fluid line,
a pair of electrically conductive contacts defining a conductivity sensor configured to measure a conductivity of fluid within the fluid conduit, and
a temperature sensor positioned generally adjacent to the pair of electrically conductive contacts and configured to measure a temperature of fluid within the fluid conduit.

11. The fluid therapy system of claim 10, wherein the sensor defines a slot, and wherein the body of the urine cartridge is configured to position the portion of the distal fluid line within the slot when coupled to the urine flow assembly.

12. The fluid therapy system of claim 10, wherein the urine flow assembly includes a receiving feature, and wherein the body of the urine cartridge includes a couple feature configured to releasably engage the receiving feature to operably engage the portion of the distal fluid line with the sensor.

13. The fluid therapy system of claim 10, wherein the portion of the distal fluid line is an upstream portion of the distal fluid line, and wherein the body of the urine cartridge defines a channel configured to receive a downstream portion of the distal fluid line.

14. The fluid therapy system of claim 10, wherein the fluid conduit has a first inner diameter, and wherein the proximal fluid line and/or the distal fluid line have a second inner diameter less than the first inner diameter.

15. The fluid therapy system of claim 10 wherein the temperature sensor is positioned upstream or downstream from one or both of the pair of electrically conductive contacts.

16. The fluid therapy system of claim 10, further comprising:
a first pump configured to provide a diuretic to the patient at a diuretic dosage rate;
a second pump configured to provide a hydration fluid to the patient at a hydration fluid infusion rate;
one or more processors; and
tangible, non-transitory computer-readable media having instructions that, when executed by the one or more processors, cause the fluid therapy system to perform operations comprising—
obtaining, via the sensor, an output rate of urine from a patient;
causing, via the first pump, a diuretic to be provided to the patient at a dosage rate;
causing, via the second pump, a hydration fluid to be provided to the patient at a hydration rate;
obtaining, via the conductivity sensor and/or the temperature sensor, one of more characteristics of the urine, wherein the one or more characteristics comprises urine conductivity; and
based on the obtained characteristics of the urine, providing an output associated with adjusting at least one of the dosage rate or the hydration rate.

17. A method for providing fluid therapy, the method comprising:
causing, via a first pump, a diuretic to be provided to a patient at a diuretic dosage rate;
causing, via a second pump, a hydration fluid to be provided to the patient at a hydration rate;
receiving, at a first end of a fluid conduit of a sensor assembly, urine from a patient via a proximal fluid line;
obtaining, via a conductivity sensor and/or a temperature sensor of the sensor assembly, one of more characteristics of the urine within the fluid conduit;
directing the urine to flow out of a second end of the fluid conduit toward a container via a distal fluid line, the second end positioned above the first end; and
based on the obtained characteristics of the urine, providing an output associated with adjusting the diuretic dosage rate and/or the hydration rate.

18. The method of claim 17, wherein directing the urine to flow out of the second end of the fluid conduit includes directing the urine to flow through an upstream portion of the distal fluid line in a first direction, and wherein the method further comprises directing the urine to flow through a downstream portion of the distal fluid line in a second direction opposite the first direction.

19. The method of claim 17, further comprising receiving an upstream portion of the distal fluid line at least partially within one or more urine line coupling features of a urine cartridge including the sensor assembly.

20. The method of claim 17, wherein the conductivity sensor includes a pair of electrically conductive contacts, and wherein obtaining the one or more characteristics includes obtaining, via the temperature sensor, a temperature of the urine between the pair of electrically conductive contacts.

21. The method of claim 17, wherein adjusting the diuretic dosage rate and/or the hydration rate includes increasing or decreasing the diuretic dosage rate and/or the hydration rate.

22. The method of claim 17, further comprising, prior to receiving the urine from the patient, flushing the sensor assembly with a solution of know electrolyte content to calibrate the conductivity sensor.
